(19) United States
(12) Patent Application Publication
(10) Pub. No.: US 2003/0068319 A1
(43) Pub. Date: Apr. 10, 2003

(54) Methods for inhibition of angiogenesis, tumor growth and metastasis by fully human anti-IL8 and anti-MUC18 in diverse types of tumors

(76) Inventor: Menashe Bar-Eli, Houston, TX (US)

Correspondence Address: FULBRIGHT & JAWORSKI, LLP, 1301 MCKINNEY, SUITE 5100, HOUSTON, TX, 77010-3095, US

(21) Appl. No.: 10/104,090

(22) Filed: Mar. 22, 2002

Related U.S. Application Data
(60) Provisional application No. 60/278,241, filed on Mar. 23, 2001.
(60) Provisional application No. 60/334,285, filed on Nov. 30, 2001.

(51) Int. Cl. A61K 39/395

(52) U.S. Cl. 424/141.1; 424/145.1

(57) ABSTRACT

The present invention relates to methods of inhibiting hyperproliferative diseases. More specifically, it concerns treating a subject suffering from a hyperproliferative disease by administering an effective amount of a human anti-IL8 antibody composition and/or a human anti-MUC18 antibody composition such that the composition inhibits the disease.

### BACKGROUND OF THE INVENTION

This application claims priority to U.S. Provisional Application No. 60/278,241, which was filed on Mar. 23, 2001 and to U.S. Provisional Application No. 60/334,285, which was filed on Nov. 13, 2001.

### FIELD OF THE INVENTION

The present invention relates generally to methods of inhibiting hyperproliferative cell growth. More particularly, it concerns the use of humanized anti-interleukin-8 antibodies and humanized anti-melanoma cell adhesion molecule antibodies for the treatment of hyperproliferative diseases.

### DESCRIPTION OF RELATED ART

Normal tissue homeostasis is a highly regulated process of cell proliferation and cell death. An imbalance of either cell proliferation or cell death can develop into a cancerous state (Solyanik et al., 1995; Stokke et al., 1997; Mumby and Walter, 1991; Natoli et al., 1998; Magi-Galluzzi et al., 1998). For example, cervical, kidney, lung, pancreatic, colorectal and brain cancer are just a few examples of the many cancers that can result (Erlandsson, 1998; Kolmel, 1998; Mangray and King, 1998; Gertig and Hunter, 1997). In fact, the occurrence of cancer is so high, that over 500,000 deaths per year are attributed to cancer in the United States alone.

Currently, there are few effective options for the treatment of many common cancer types. The course of treatment for a given individual depends on the diagnosis, the stage to which the disease has developed and factors such as age, sex and general health of the patient. The most conventional options of cancer treatment are surgery, radiation therapy and chemotherapy. Surgery plays a central role in the diagnosis and treatment of cancer. Typically, a surgical approach is required for biopsy and to remove cancerous growth. However, if the cancer has metastasized and is widespread, surgery is unlikely to result in a cure and an alternate approach must be taken. Radiation therapy, chemotherapy and immunotherapy are alternatives to surgical treatment of cancer (Mayer, 1998; Ohara, 1998).

Immunotherapy is a rapidly evolving area in cancer research. However, administration of antibodies in medical therapy can be problematic because antibodies displaying non-human characteristics tend to prompt immunogenic responses in humans. Such responses necessarily interfere with the efficacy of the therapy and may promote unintended effects, further harming the patient. However, fully human antibodies may be made so that the undesirable immunogenic responses are minimized or avoided.

### SUMMARY OF THE INVENTION

The present invention overcomes deficiencies in the art by providing an anti-cancer therapy that modulates interleukin-8 (IL-8) activity and/or melanoma cell adhesion molecule (MCAM/MCU18) activity in a variety of cancers through the use of fully human antibodies specific to IL-8 and/or MUC18. It is envisioned that the antibodies to IL-8 and MUC18 bind to IL-8 and MUC18, respectively, and thereby disrupt the normal role of IL-8 and MUC18 in tumor growth and metastasis. It is envisioned that humanized or fully human antibodies modulate activity without detrimental side effects associated with administration of non-human antibodies to human subjects.

One aspect of the present invention encompasses administrating to a patient humanized antibodies specific for IL-8 to modulate IL-8 activity in hyperproliferative disease or disorder. An additional embodiment encompasses the administration of such anti-IL-8 antibodies that are fully human. In a further embodiment, the invention concerns treatment of a patient in need of such treatment through administration of fully human anti-IL-8 antibodies directed towards the inhibition, reduction or prevention of the proliferation of cancer cells. A further embodiment of the present invention concerns the use of chemotherapeutic agents, anti-angiogenic agents, or gene therapy as an adjunct to such antibody treatment. The patient is preferably a mammal and more preferably a human. Yet further, the patient suffers from a hyperproliferative disease or disorder.

A further aspect of the present invention encompasses the administrating to a patient humanized antibodies specific for MUC18 to modulate MUC18 activity in hyperproliferative disease or disorder. An additional embodiment encompasses the administration of such anti-MUC18 antibodies that are fully human. The MUC18 antibodies may be MUC18:A15 6.9, MUC18:A15 6.11 or MUC18:A153.19. In a further embodiment, the invention concerns treatment of a patient in need of such treatment through administration of fully human anti-MUC18 antibodies directed towards the inhibition, reduction or prevention of the proliferation of cancer cells. A further embodiment of the present invention concerns the use of chemotherapeutic agents, anti-angiogenic agents, or gene therapy as an adjunct to such antibody treatment.

Yet further, the present invention concerns the use of a combination of anti-MUC18 antibodies and anti-IL-8 antibodies to treat hyperproliferative diseases or disorders. It is also envisioned that anti-MUC18 antibodies and/or anti-IL-8 antibodies may also be used in conjunction with other anti-cancer therapies, such as chemotherapeutic agents, radiation or surgery.

In certain embodiments, the hyperproliferative disease is further defined as cancer. In still further embodiments, the cancer is melanoma, non-small cell lung, small-cell lung, lung, hepatocarcinoma, leukemia, retinoblastoma, astrocytoma, glioblastoma, gum, tongue, neuroblastoma, head, neck, breast, pancreatic, prostate, renal, bone, testicular, ovarian, mesothelioma, cervical, gastrointestinal, lymphoma, brain, colon, sarcoma or bladder. The cancer may include a tumor comprised of tumor cells. For example, tumor cells may include, but are not limited to melanoma cell, a bladder cancer cell, a breast cancer cell, a lung cancer cell, a colon cancer cell, a prostate cancer cell, a liver cancer cell, a pancreatic cancer cell, a stomach cancer cell, a testicular cancer cell, a brain cancer cell, an ovarian cancer cell, a lymphatic cancer cell, a skin cancer cell, a brain cancer cell, a bone cancer cell, or a soft tissue cancer cell.

In other embodiments, the hyperproliferative disease is rheumatoid arthritis, inflammatory bowel disease, osteoarthritis, leiomyomas, adenomas, lipomas, hemangiomas, fibromas, vascular occlusion, restenosis, atherosclerosis, pre-neoplastic lesions (such as adenomatous hyperplasia and prostatic intraepithelial neoplasia), carcinoma in situ, oral hairy leukoplakia, or psoriasis.

The present invention also relates to methods of using human anti-IL-8 antibodies and/or human anti-MUC18 antibodies to inhibit or prevent angiogenesis due to secretion of IL-8 and/or expression of MUC18 by melanoma cells in a subject. It is envisioned that administration of anti-IL-8 antibodies, anti-MUC18 antibodies, a combination of both antibodies, or one or both antibodies in combination with an anti-cancer agent may inhibit or prevent tumor growth of human melanoma cells or tumors in a subject; inhibit or prevent metastasis of human melanoma cells or tumors in a subject; inhibit or prevent type IV collagenases (matrix metalloproteinase-2 (MMP-2) or matrix metalloproteinase-9 (MMP-9)) expression in a subject; reduce metastatic potential of human melanoma cells or tumors; or induce apoptosis of IL-8 expressing melanoma cells or tumors in a subject; or MUC18 expressing melanoma cells or tumors in a subject.

Each of the methods of this invention comprises the step of administering an effective amount of an antibody, or antigen binding fragment thereof, to a subject in need of such administration; preferably the human antibody is a human monoclonal antibody; even more preferably the human monoclonal antibody is a monoclonal antibody produced by a transgenic mouse that produces human antibodies preferably to the exclusion of murine antibodies; even more preferably the human monoclonal antibody is a monoclonal antibody produced by a XENOMOUSE™ animal.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of illustrative embodiments presented herein.

FIG. 1 shows the proliferation of control and ABX-IL8 treated melanoma cells in vitro.

FIG. 2 shows the amount of IL-8 secretion by melanoma cells after treatment with ABX-IL8.

FIG. 3 illustrates the rate of tumor growth in vivo in control animals and animals treated with ABX-IL8.

FIG. 4 illustrates the activity of MMP-2 in control and ABX-IL8 treated melanoma cells.

FIG. 5A, FIG. 5B, FIG. 5C and FIG. 5D illustrate angiogenesis and apoptosis in control and ABX-IL8 treated cells. FIG. 5A and FIG. 5B show angiogenesis in control (FIG. 5A) and ABX-IL8 treated (FIG. 5B) cells. Apoptosis is measured by the tunnel assay in control (FIG. 5C) and ABX-IL8 treated (FIG. 5D) cells.

FIG. 6 shows the cell proliferation in control and ABX-IL8 treated bladder cancer cells.

FIG. 7 shows the amount of IL8 expression after transfection of pcDNA-sense IL8.

FIG. 8A and FIG. 8B show the ability of ABX-IL8 to neutralize IL8 in bladder cancer cells.

FIG. 9A FIG. 9B FIG. 9G and FIG. 9D show a decrease in MMP-9 and MMP-2 activity in a variety of bladder cancer cells; 253J-P (FIG. 9A), 253J-BV (FIG. 9C) 253J-Neo (FIG. 9B) and 253J-SIL8 (FIG. 9D).

FIG. 10 shows the amount of luciferase activity, which is a measure of promoter activity.

FIG. 11A and FIG. 11B show an analysis of MUC18/MCAM in melanoma cells. FIG. 11A shows a Western blot analysis of MUC18/MCAM in melanoma cells and HUVEC by ABX-MUC18. FIG. 11B shows a FACS analysis on the cell surface of A375SM cells.

FIG. 12 shows disruption of spheroid formation by ABX-MUC18.

FIG. 13A and FIG. 13B show the effect of ABX-MUC18 on tumor growth of human melanoma cells in nude mice.

FIG. 14A, FIG. 14B, and FIG. 14C show an effect of ABX-MUC18 on proliferation of melanoma and HUVEC in vitro.

FIG. 15 shows MMP-2 activity in control and anti-MUC18 treated melanoma cells.

FIG. 16A and FIG. 16B show the downregulation of MMP-2 activity in melanoma cells by ABX-MUC18. Fold decrease in luciferase activity was calculated relative to the luciferase activity in untreated cells, which was assigned the value of 1.

FIG. 17 shows the percent melanoma cells to that adhered to HUVECS.

FIG. 18A and FIG. 18B show an effect of ABX-MUC18 on MMP-2 expression in vivo.

FIG. 19 shows the amount of cell invasion in control and anti-MUC18 treated cells.

FIG. 20A and FIG. 20B show a tumor microvessel density (MVD) (FIG. 20A) and apoptosis (TUNEL) (FIG. 20B) in subcutaneous melanoma xenografts.

FIG. 21A and FIG. 21B show an effect of ABX-MUC18 on attachment of melanoma cells to HUVEC. FIG. 21B shows the interaction of A375SM with HUVEC is summarized in bars graph and demonstrates that ABX-MUC18 inhibited A375SM-HUVEC interaction by 80%.

FIG. 22A, FIG. 22B, FIG. 22C and FIG. 22D show an effect of ABX-MUC18 on vessel-like tube formation by HUVEC. FIG. 22A shows untreated HUVEC cells formed the tube like vessels; FIG. 22B shows treatment with control IgG did not disrupt the vessel-like tube formation; FIG. 22C shows ABX-MUC18 pretreatment disrupted vessel-like tube formation by HUVEC; and FIG. 22D shows no effect was observed when ABX-MUC18 was added after the network had been established.

### DESCRIPTION OF ILLUSTRATIVE EMBODIMENTS

Therapeutic regimens employed in the therapy of solid tumor malignancies have not proven very effective in late stage disease. For example, although melanoma is often curable when diagnosed and surgically treated at an early stage, most patients with metastatic melanoma die of their disease due to absence of effective therapy. Aggressive cytotoxic regimens with their attendant morbidity have been unable to qualitatively and quantitatively impact on patients with metastatic disease. Thus, there is a great need for new therapies effective against solid tumor cancers. In particular, there is a need for therapies that have reduced toxicity but are nevertheless effective against the growth, angiogenesis, and metastasis of solid tumors.

A. Melanoma Cells

Melanoma cells secrete a variety of growth factors either constitutively or subsequent to induction by other cytokines (Matte 1994; Kerbel 1992; Herlyn, 1990). Among the cytokines secreted by melanoma cells are transforming growth factor-alpha (Fidler and Kripke, 1977), transforming growth factor-Beta (Fidler 1992), platelet-derived growth factors A and B (Luca et al., 1993), basic bifroblast growth factor (bFGF) (Xie et al., 1997), interleukin (IL)-1 (Huang et al., 1996; Singh et al., 1995), IL-6, (Mastrangelo et al., 1985; Luca et al., 1997), IL-10 (Xie et al., 1997; Bar-Eli, 1997), granulocyte/macrophage colony-stimulating factor, (Luca et al., 1993) and melanoma growth-stimulating activity (MGSA) (Fidler and Kripke, 1977). These growth factors/cytokines may act as autocrine growth factors or act in paracrine fashion on the host environment to stimulate growth (Price et al., 1986).

The development of malignant melanoma in humans progresses through a multistage process. The switch from melanocyte to nevi, to radial growth and subsequently to vertical growth phase (metastatic phenotype) are associated with decreased dependence on growth factors, diminished anchorage dependence, and reduced contact inhibition (O'Rourke and Altman, 1993; Fidler, 1990; Lu and Kerbel, 1994). The growth and progression of melanoma cells require autocrine growth factor production and the ability to respond to external stimuli such as hormones or growth and differentiation factors (Radinsky, 1991).

Increased proliferation, however, is not enough to give rise to metastases. To possess metastatic potential, a cell has to be able to invade the surrounding tissue, spread via lymphatics and/or the bloodstream, extravasate, and multiply at a secondary site. Genes involved in cell attachment, motility, and proteolytic degradation of the extracellular matrix are important in these processes.

B. The Role of IL-8 in Cancer

Interleukin-8 (IL-8) was originally identified as a leukocyte chemoattractant and it shares 44% amino acid homology with MGSA/gro, an autocrine growth factor for melanoma cells (Gutman et al, 1994; Luca et al., 1995). IL-8 has been shown to induce both angiogenesis and haptotactic migration in melanoma cells (Huang et al., 1996, Jean et al., 1996; Lu and Kerbel 1994). Constitutive expression of IL-8 in human melanoma cells directly correlates with their metastatic potential in nude mice and UV-B irradiation of primary cutaneous melanoma cells rendered them highly tumorigenic and increased their metastic potential via the induction of IL-8 (Radinsky 1991; Matte et al., 1994).

IL-8 has been shown to be an angiogenic factor, and angiogenesis is a known requirement of the growth and metastasis of solid cancers. Angiogenesis results in the vascularization of the solid tumor. A vascular supply system is required for continued growth and proliferation of such solid tumors. IL-8 may exert its angiogenic activity by upregulating the expression and activity of MMP-2, which is the major active metalloproteinase in human melanoma. Activation of type IV collagenase (MMP-2) by IL-8 can enhance the invasion of host stroma by the tumor cells and enhance angiogenesis, and hence metastasis.

The type IV collagenase/gelatinase MMP-2 and MMP-9 are involved in a wide array of biological activities, including invasion, metastasis and angiogenesis. The ability of tumor cells to degrade connective-tissue extracellular matrix (ECM) and basement membrane component is an essential prerequisite for invasion and metastasis. Increased expression of MMP-2 has been demonstrated in metastatic melanoma cells compared to non-metastatic counterparts and that of normal mucosa cells. In the process of angiogenesis, endothelial cells must invade through the ECM toward the source of the angiogenic stimulus. Proteolysis of the ECM by MMPs allows for endothelial cell migration and may also release sequestered signaling molecules such as bFGF. MMPs are implicated in this process by their expression in and around forming blood vessels and by the observation that MMPs-deficient mice demonstrated delayed release of angiogenic activators.

Thus, IL-8 plays a key role in regulating cell growth and metastasis of solid cancer cells. Modulation of IL-8's effects on this system, thus, can inhibit cancer cell proliferation and metastasis. Thus, the present inventor contemplates the use of human anti-interleukin-8 antibody to treat patients with melanoma either alone or in combination with chemotherapy or in combination with another immunotherapy. Yet further, the present invention may also be used in combination with other anti-cancer agents, such as radiation or surgery.

C. Role of MUC18 in Cancer

MUC18 (MCAM), is a cell surface adhesion molecule that is strongly expressed by advanced primary and metastic melanomas and is express less frequent in nevus cells. It has been demonstrated that MUC18 expression correlates with the metastatic potential of human melanoma cells in nude mice. Yet further, it has been shown that enforced expression of MUC18 in primary cutaneous melanoma (MUC18-negative) resulted in an increase in tumor growth and metastatic potential in vivo. The transfected cells displayed increased homotypic adhesion, increased attachment to human endothelial cells, decreased ability to adhere to laminin, and upregulation of metalloproteinase-2 (MMP-2) which resulted in increased invasiveness through Matrigel-coated filters. MUC18 is also expressed on vascular endothelial cells that are involved in angiogenesis.

Thus, the present inventor contemplates the use of human anti-MUC18 antibody to treat patients with melanoma either alone or in combination with chemotherapy or in combination with another immunotherapy. Yet further, the present invention may also be used in combination with other anti-cancer agents, such as radiation or surgery.

D. Antibodies for Immunotherapy

The present invention provides antibodies to be used as an immunotherapy for hyperproliferative diseases and disorders. The antibodies of the present invention are immunoreactive with human IL-8 or human MUC18 and are compatible with the human immune system so as to be less immunogenic to humans than the murine monoclonal antibodies presently available. The invention also provides intermediates for the preparation of these antibodies or their immunoreactive fragments. Thus, the invention provides proteins, which comprise the variable regions of the heavy and light chains of antibodies which are immunospecific for human IL-8 or human MUC18 wherein these variable regions have human characteristics. In general, these variable regions contain the complementarity determining regions (CDRs) of the pertinent antibody secreted by nonhuman cells but the framework regions (FRs) which characterize human antibodies. By thus reshaping murine- or other nonhuman-derived immunoglobulins, their immunogenicity in human patients is minimized.

Thus, in one aspect, the invention is directed to a humanized monoclonal antibody immunoreactive with human IL-8 or human MUC18 wherein the framework regions (FRs) of the variable regions of said antibody and the constant regions of said antibody are compatible with the human immune system. More specifically, the monoclonal antibody or immunoreactive fragment thereof, is immunoreactive with human IL-8 or human MUC18 and compatible with the human immune system, wherein the framework regions (FRs) of the variable regions of said antibody or fragment and any constant regions of said antibody or fragment are of human origin.

Thus, as used herein the term “humanized” is directed to antibodies or fragments immunospecific for human IL-8 or human MUC18 which have sufficient human characteristics so that their immunogenicity in human systems is lowered with respect to the corresponding antibodies derived from other species. Thus, the humanized antibodies or immunoreactive fragments of the invention are compatible with the human immune system. By “compatible with the human immune system” is meant that the antibodies or fragments of the invention do not elicit a substantial immune response when administered to humans as compared to unmodified forms of nonhuman antibodies containing the same complementarity-determining regions (CDRs). Eliciting an immune response is clearly undesirable as antibodies raised against therapeutically administered materials undermine the effectiveness of the administered materials and in addition may provoke unwanted side-effects due to stimulation of the immune system per se. While the antibodies and fragments of the invention may not, of course, be completely neutral with respect to an immune response in a specific individual, their effect on the immune system of an individual will be substantially less than that elicited by corresponding nonhuman antibodies in their unmodified forms.

Yet further, as used herein, the term “fully human antibody” or “fully humanized antibody” refers to antibodies or fragments immunospecific for human IL-8 or human MUC18 which have relatively no CDR or FR residues substituted from analogous sites in nonhuman species. Thus, the human variable doman is intact.

As discussed herein, minor variations in the amino acid sequences of antibodies or immunoglobulin molecules are contemplated as being encompassed by the humanized antibodies of the present invention, providing that the variations in the amino acid sequence maintain at least 75%, more preferably at least 80%, 90%, 95%, and most preferably 99% homology to the human variable domain. Specifically, in the present invention if the humanized antibody maintains at least 95% and most preferably 99% homology to the human variable domain, then the humanized antibody is considered to be fully humanized.

In particular, the variations that may be contemplate are conservative amino acid replacements. Conservative replacements are those that take place within a family of amino acids that are related in their side chains. Genetically encoded amino acids are generally divided into families: (1) acidic=aspartate, glutamate; (2) basic=lysine, arginine, histidine; (3) non-polar=alanine, valine, leucine, isoleucine, proline, phenylalanine, methionine, tryptophan; and (4) uncharged polar=glycine, asparagine, glutamine, cysteine, serine, threonine, tyrosine. More preferred families are: serine and threonine are aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Fragments or analogs of antibodies or immunoglobulin molecules can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known (Bowie et al., 1991).

Preferred amino acid substitutions are those which: (1) reduce susceptibility to proteolysis, (2) reduce susceptibility to oxidation, (3) alter binding affinity for forming protein complexes, (4) alter binding affinities, and (4) confer or modify other physiocochemical or functional properties of such analogs. Analogs can include various mutations of a sequence other than the naturally-occurring peptide sequence. For example, single or multiple amino acid substitutions (preferably conservative amino acid substitutions) may be made in the naturally-occurring sequence (preferably in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterizes the parent sequence).

In certain aspects of the invention, one or more of the antibodies may be a commercially available therapeutic antibody. For example, but not limited to, ABX-IL8 and ABX-MUC18. In specific embodiments of the present invention, IL-8 and MUC18 are humanized antibodies. ABX-IL8 is a fully human IgG2 monoclonal antibody directed against IL8. ABX-IL8 is described in more detail in International Patent Application WO 98/24893, published Jun. 11, 1998, and U.S. patent application Ser. No. 09/203,268, filed Dec. 10, 1998, the disclosures of both of which are hereby incorporated by reference. MUC18 may also be a fully humanized anti-MUC18 antibody, such as ABX-MUC18. These antibodies may be used in various therapeutic applications, described herein below.

The present invention also relates to a pharmaceutical composition comprising an anti-IL-8 compound; preferably the anti-IL-8-compound is an anti-IL-8 antibody; more preferably, the anti-IL-8 antibody is a human anti-IL-8 antibody; more preferably, the human anti-IL-8 antibody is a human monoclonal antibody; even more preferably the human anti-IL-8 monoclonal antibody produced by a transgenic mouse that produces human antibodies preferably to the exclusion of murine antibodies; even more preferably the human anti-IL-8 monoclonal antibody is a monoclonal antibody produced by a XENOMOUSE™ animal; and even more preferably the human anti-IL8 monoclonal antibody produced by a XENOMOUSE™ animal is ABX-IL8.

In a further embodiment, the present invention also relates to a pharmaceutical composition comprising an anti-MUC18 compound; preferably the anti-MUC18-compound is an anti-MUC18 antibody; more preferably, the anti-MUC18 antibody is a human anti-MUC18 antibody; more preferably, the human anti-MUC18 antibody is a human monoclonal antibody; even more preferably the human anti-MUC18 monoclonal antibody produced by a transgenic mouse that produces human antibodies preferably to the exclusion of murine antibodies; even more preferably the human anti-MUC18 monoclonal antibody is a monoclonal antibody produced by a XENOMOUSE™ animal; and even more preferably the human anti-MUC18 monoclonal antibody produced by a XENOMOUSE™ animal is ABX-MUC18.

E. Antibody Preparation

Yet further, the antibodies of the present invention may be produced using standard procedures that are well known and used in the art.

1. Polyclonal Antibodies

Polyclonal antibodies to IL-8 and MUC18 generally are raised in animals by multiple subcutaneous (sc) or intraperitoneal (ip) injections of the IL-8 and MUC18 and an adjuvant.

Animals are immunized against the immunogenic composition or derivatives. Animals are boosted until the titer plateaus. The animals are usually bled through an ear vein or alternatively by cardiac puncture. The removed blood is allowed to coagulate and then centrifuged to separate serum components from whole cells and blood clots. The serum may be used as is for various applications or else the desired antibody fraction may be purified by well-known methods, such as affinity chromatography using another antibody, a peptide bound to a solid matrix, or by using, e.g., protein A or protein G chromatography.

2. Monoclonal Antibodies

The methods for generating monoclonal antibodies (MAbs) generally begin along the same lines as those for preparing polyclonal antibodies. Rodents such as mice and rats are preferred animals, however, the use of rabbit, sheep, goat, monkey cells also is possible. The use of rats may provide certain advantages (Goding, 1986), but mice are preferred, with the BALB/c mouse being most preferred as this is most routinely used and generally gives a higher percentage of stable fusions.

Monoclonal antibodies are obtained from a population of substantially homogeneous antibodies, i.e., the individual antibodies comprising the population are identical except for possible naturally-occurring mutations that may be present in minor amounts. Thus, the modifier “monoclonal” indicates the character of the antibody as not being a mixture of discrete antibodies.

The animals are injected with antigen, generally as described above for polyclonal antibodies. The antigen may be coupled to carrier molecules such as keyhole limpet hemocyanin if necessary. The antigen would typically be mixed with adjuvant, such as Freund's complete or incomplete adjuvant. Booster injections with the same antigen would occur at approximately two-week intervals.

Following immunization, somatic cells with the potential for producing antibodies, specifically B-lymphocytes (B cells), are selected for use in the MAb generating protocol. These cells may be obtained from biopsied spleens or lymph nodes. Spleen cells and lymph node cells are preferred, the former because they are a rich source of antibody-producing cells that are in the dividing plasmablast stage.

Often, a panel of animals will have been immunized and the spleen of animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B-lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Methods for generating hybrids of antibody-producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. Fusion methods using Sendai virus have been described by Kohler and Milstein (1976), and those using polyethylene glycol (PEG), such as 37% (v/v) PEG. The use of electrically induced fusion methods also is appropriate (Goding, 1986).

Fusion procedures usually produce viable hybrids at low frequencies, about 1×10⁻⁶ to 1×10⁻⁸. However, this does not pose a problem, as the viable, fused hybrids are differentiated from the parental, infused cells particularly the infused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays, dot immunobinding assays, and the like.

The selected hybridomas would then be serially diluted and cloned into individual antibody-producing cell lines, which clones can then be propagated indefinitely to provide MAbs. The cell lines may be exploited for MAb production in two basic ways.

A sample of the hybridoma can be injected (often into the peritoneal cavity) into a histocompatible animal of the type that was used to provide the somatic and myeloma cells for the original fusion (e.g., a syngeneic mouse). Optionally, the animals are primed with a hydrocarbon, especially oils such as pristane (tetramethylpentadecane) prior to injection. The injected animal develops tumors secreting the specific monoclonal antibody produced by the fused cell hybrid. The body fluids of the animal, such as serum or ascites fluid, can then be tapped to provide MAbs in high concentration.

The individual cell lines could also be cultured in vitro, where the MAbs are naturally secreted into the culture medium from which they can be readily obtained in high concentrations.

MAbs produced by either means may be further purified, if desired, using filtration, centrifugation and various chromatographic methods such as HPLC or affinity chromatography. Fragments of the monoclonal antibodies of the invention can be obtained from the purified monoclonal antibodies by methods, which include digestion with enzymes, such as pepsin or papain, and/or by cleavage of disulfide bonds by chemical reduction. Alternatively, monoclonal antibody fragments encompassed by the present invention can be synthesized using an automated peptide synthesizer.

It also is contemplated that a molecular cloning approach may be used to generate monoclonals. For this, combinatorial immunoglobulin phagemid libraries are prepared from RNA isolated from the spleen of the immunized animal, and phagemids expressing appropriate antibodies are selected by panning using cells expressing the antigen and control cells e.g., normal-versus-tumor cells. The advantages of this approach over conventional hybridoma techniques are that approximately 104 times as many antibodies can be produced and screened in a single round, and that new specificities are generated by H and L chain combination which further increases the chance of finding appropriate antibodies.

3. Humanized Antibodies

Methods for humanizing non-human antibodies are well known in the art. Generally, a humanized antibody has one or more amino acid residues introduced into it from a source, which is non-human. These non-human amino acid residues are often referred to as “import” residues, which are typically taken from an “import” variable domain. Humanization can be essentially performed following the method of Winter and co-workers (Jones et al., 1986; Riechmann et al., 1988; Verhoeyen et al., 1988), by substituting rodent CDRs or CDR sequences for the corresponding sequences of a human antibody. Accordingly, such “humanized” antibodies are chimeric antibodies wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

It is important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e. the analysis of residues that influence the ability of the candidate immunoglobulin to bind its antigen.

4. Human Antibodies

Human monoclonal antibodies can be made by the hybridoma method. Human myeloma and mouse-human heteromyeloma cell lines for the production of human monoclonal antibodies have been described (Kozbor, 1984; U.S. Pat. No. 6,150,584, which is incorporated herein by reference).

It is now possible to produce transgenic animals (e.g., mice) that are capable, upon immunization, of producing a repertoire of human antibodies in the absence of endogenous immunoglobulin production. For example, it has been described that the homozygous deletion of the antibody heavy chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. (Jakobovits et al., 1993).

Alternatively, the phage display technology (McCafferty et al., 1990) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle.

F. Immunotherapy Treatments

1. Treatment of Hyperproliferative Diseases

In certain embodiments, a hyperproliferative disease may be treated by administering to a subject an effective amount of anti-IL-8 and/or anti-MUC18 antibodies. The subject is preferably a mammal and more preferably a human.

In the present invention, a hyperproliferative disease is further defined as cancer. In still further embodiments, the cancer is melanoma, non-small cell lung, small-cell lung, lung, leukemia, hepatocarcinoma, retinoblastoma, astrocytoma, glioblastoma, gum, tongue, neuroblastoma, head, neck, breast, pancreatic, prostate, renal, bone, testicular, ovarian, mesothelioma, cervical, gastrointestinal, lymphoma, brain, colon, sarcoma or bladder.

The cancer may include a tumor comprised of tumor cells. For example, tumor cells may include, but are not limited to melanoma cell, a bladder cancer cell, a breast cancer cell, a lung cancer cell, a colon cancer cell, a prostate cancer cell, a liver cancer cell, a pancreatic cancer cell, a stomach cancer cell, a testicular cancer cell, a brain cancer cell, an ovarian cancer cell, a lymphatic cancer cell, a skin cancer cell, a brain cancer cell, a bone cancer cell, or a soft tissue cancer cell.

In other embodiments, the hyperproliferative disease is rheumatoid arthritis, inflammatory bowel disease, osteoarthritis, leiomyomas, adenomas, lipomas, hemangiomas, fibromas, vascular occlusion, restenosis, atherosclerosis, pre-neoplastic lesions (such as adenomatous hyperplasia and prostatic intraepithelial neoplasia), carcinoma in situ, oral hairy leukoplakia, or psoriasis.

In a preferred embodiment of the present invention, anti-IL-8 and/or anti-MUC18 antibodies are administered in an effective amount to decrease, reduce, inhibit or abrogate the growth of a solid tumor. Examples of solid tumors that can be treated according to the invention include sarcomas and carcinomas such as, but not limited to: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

Yet further, hyperproliferative diseases that are most likely to be treated in the present invention are those that metastasize. It is understood by those in the art that metastasis is the spread of cells from a primary tumor to a noncontiguous site, usually via the bloodstream or lymphatics, which results in the establishment of a secondary tumor growth. Examples of hyperproliferative diseases contemplated for treatment include, but are not limited to melanoma, bladder, non-small cell lung, small cell lung, lung, hepatocarcinoma, retinoblastoma, astrocytoma, glioblastoma, neuroblastoma, head, neck, breast, pancreatic, gum, tongue, prostate, renal, bone, testicular, ovarian, mesothelioma, cervical, gastrointestinal lymphoma, brain, or colon cancer and any other hyperproliferative diseases that may be treated by administering a an anti-IL-8 and/or anti-MUC18 antibody.

2. Treatment Regimens

Treatment regimens may vary as well, and often depend on tumor type, tumor location, disease progression, and health and age of the patient. Obviously, certain types of tumor will require more aggressive treatment, while at the same time, certain patients cannot tolerate more taxing protocols. The clinician will be best suited to make such decisions based on the known efficacy and toxicity (if any) of the therapeutic formulations.

Preferably, patients to be treated will have adequate bone marrow function (defined as a peripheral absolute granulocyte count of >2,000/mm³ and a platelet count of 100,000/mm³), adequate liver function (bilirubin<1.5 mg/dl) and adequate renal function (creatinine<1.5 mg/dl).

As used herein the term “effective amount” is defined as an amount of the agent that will decrease, reduce, inhibit or otherwise abrogate the growth of a cancer cell, induce apoptosis, inhibit angiogenesis of a tumor cell, inhibit metastasis, or induce cytotoxicity in cells. Thus, an effective amount is an amount sufficient to detectably and repeatedly ameliorate, reduce, minimize or limit the extent of the disease or its symptoms. More rigorous definitions may apply, including elimination, eradication or cure of disease.

To kill cells, inhibit cell growth, inhibit metastasis, decrease tumor or tissue size and otherwise reverse or reduce the malignant phenotype of tumor cells, using the methods and compositions of the present invention, one would generally contact a hyperproliferative cell with the anti-IL-8 and/or anti-MUC18 antibody. The routes of administration will vary, naturally, with the location and nature of the lesion, and include, e.g., intradernal, transdermal, parenteral, intravenous, intramuscular, intranasal, subcutaneous, percutaneous, intratracheal, intraperitoneal, intratumoral, perfusion, lavage, direct injection, and oral administration and formulation.

In the case of surgical intervention, the present invention may be used preoperatively, to render an inoperable tumor subject to resection. Alternatively, the present invention may be used at the time of surgery, and/or thereafter, to treat residual or metastatic disease. For example, a resected tumor bed may be injected or perfused with a formulation comprising anti-IL-8 and/or anti-MUC18 antibody. The perfusion may be continued post-resection, for example, by leaving a catheter implanted at the site of the surgery. Periodic post-surgical treatment also is envisioned.

Continuous administration also may be applied where appropriate, for example, where a tumor is excised and the tumor bed is treated to eliminate residual, microscopic disease. Delivery via syringe or catherization is preferred. Such continuous perfusion may take place for a period from about 1-2 hours, to about 2-6 hours, to about 6-12 hours, to about 12-24 hours, to about 1-2 days, to about 1-2 wk or longer following the initiation of treatment. Generally, the dose of the therapeutic composition via continuous perfusion will be equivalent to that given by a single or multiple injections, adjusted over a period of time during which the perfusion occurs. It is further contemplated that limb perfusion may be used to administer therapeutic compositions of the present invention, particularly in the treatment of melanomas and sarcomas.

In certain embodiments, the tumor being treated may not, at least initially, be resectable. Treatments with therapeutic antibodies may increase the resectability of the tumor due to shrinkage at the margins or by elimination of certain particularly invasive portions. Following treatments, resection may be possible. Additional treatments subsequent to resection will serve to eliminate microscopic residual disease at the tumor site.

A typical course of treatment, for a primary tumor or a post-excision tumor bed, will involve multiple doses. Typical primary tumor treatment involves a 6 dose application over a two-week period. The two-week regimen may be repeated one, two, three, four, five, six or more times. During a course of treatment, the need to complete the planned dosings may be re-evaluated.

The treatments may include various “unit doses.” Unit dose is defined as containing a predetermined-quantity of the therapeutic composition. The quantity to be administered, and the particular route and formulation, are within the skill of those in the clinical arts. A unit dose need not be administered as a single injection but may comprise continuous infusion over a set period of time.

3. Treatment Regimen for Melanoma

It is envisioned that melanoma may be treated by employing the antibody treatment of the present invention. For example, ABX-IL8 and/or ABX-MUC18 may be employed at a starting dose of 1-3 mg/kg. Dosing may be every 3 weeks for 4 cycles (total=12 weeks), at which time response may also be determined. If no dose-limiting toxicity is observed after 2 cycles, then the next dosing level may be initiated according to standard dose-escalation algorithms (i.e., 3 mg/kg, 6 mg/kg, 9 mg/kg, 13.5 mg/kg, etc.).

In addition to toxicity and response data, tissue and serum samples are collected pre-therapy and post-therapy (after 2 and 4 cycles) to provide the basis for studies on intermediate biomarkers involved in angiogenesis and invasion and to evaluate whether these markers can predict response to treatment. To assess for alterations in blood flow, in situ, blood flow patterns are assessed in real time using 3-D re-constructions of high resolution cutaneous Doppler ultrasound examinations of accessible tumors pre-therapy and after 2 to 4 cycles.

G. Combination Treatments

In order to increase the effectiveness of either anti-IL-8 or anti-MUC18, it may be desirable to combine these compositions and administer both antibodies to the patient. Yet further, it may be desirable to combine either anti-IL-8 or anti-MUC18 with other agents effective in the treatment of hyperproliferative disease, such as anti-cancer agents, or with surgery. It is also contemplated that both anti-IL-8 and anti-MUC18 may be administered in combination with an additional anti-cancer agent. An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. Anti-cancer agents include biological agents (biotherapy), chemotherapy agents, and radiotherapy agents. More generally, these other compositions would be provided in a combined amount effective to kill or inhibit proliferation of the cell. This process may involve contacting the cells with the antibodies of the present invention and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the antibodies and the other includes the second agent(s).

Alternatively, the antibodies of the present invention (anti-IL8 and/or anti-MUC18) may precede or follow the other anti-cancer agent treatment by intervals ranging from minutes to weeks. In embodiments where the other anti-cancer agent and antibodies are applied separately to the cell, one would generally ensure that a significant period of time did not expire between the time of each delivery, such that the agent and antibodies would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations.

Various combinations may be employed, antibodies is “A” and the secondary agent, such as radio- or chemotherapy, is “B”:

A/B/A B/A/B B/B/A A/A/B A/B/B B/A/A A/B/B/B
B/A/B/B B/B/B/A B/B/A/B A/A/B/B A/B/A/B A/B/B/A
B/B/A/A B/A/B/A B/A/A/B A/A/A/B B/A/A/A A/B/A/A
A/A/B/A

Administration of the immunotherapy of the present invention to a patient will follow general protocols for the administration of chemotherapeutics. It is expected that the treatment cycles would be repeated as necessary. It also is contemplated that various standard therapies, as well as surgical intervention, may be applied in combination with the described hyperproliferative cell therapy.

1. Chemotherapy

Cancer therapies also include a variety of chemical based treatments. Some examples of chemotherapeutic agents include antibiotic chemotherapeutics such as Doxorubicin, Daunorubicin, Adriamycin, Mitomycin (also known as mutamycin and/or mitomycin-C), Actinomycin D (Dactinomycin), Bleomycin, Plicomycin, plant alkaloids such as Taxol, Vincristine, Vinblastine, miscellaneous agents such as Cisplatin (CDDP), etoposide (VP16), Tumor Necrosis Factor, and alkylating agents such as, Carmustine, Melphalan (also known as alkeran, L-phenylalanine mustard, phenylalanine mustard, L-PAM, or L-sarcolysin, is a phenylalanine derivative of nitrogen mustard), Cyclophosphamide, Chlorambucil, Busulfan (also known as myleran), Lomustine.

Some examples of other agents include, but are not limited to, Carboplatin, Procarbazine, Mechlorethamine, Camptothecin, Ifosfamide, Nitrosurea, Etoposide (VP16), Tamoxifen, Raloxifene, Toremifene, Idoxifene, Droloxifene, TAT-59, Zindoxifene, Trioxifene, ICI 182,780, EM-800, Estrogen Receptor Binding Agents, Gemcitabien, Navelbine, Farnesyl-protein transferase inhibitors, Transplatinum, 5-Fluorouracil, hydrogen peroxide, and Methotrexate, Temazolomide (an aqueous form of DTIC), Mylotarg, Dolastatin-10, Bryostatin, or any analog or derivative variant of the foregoing.

2. Radiotherapeutic Agents

Radiotherapeutic agents and factors include radiation and waves that induce DNA damage for example, γ-irradiation, X-rays, UV-irradiation, microwaves, electronic emissions, radioisotopes, and the like. Therapy may be achieved by irradiating the localized tumor site with the above described forms of radiations. It is most likely that all of these factors effect a broad range of damage DNA, on the precursors of DNA, the replication and repair of DNA, and the assembly and maintenance of chromosomes.

Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

3. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

4. Gene Therapy

In yet another embodiment, gene therapy in conjunction with the combination therapy using the antibody compounds described in the invention are contemplated. A variety of proteins are encompassed within the invention, some of which are described below. Table 1 lists various genes that may be targeted for gene therapy of some form in combination with the present invention.

TABLE 1
Oncogenes

| | Source | Human Disease | Function |
|---|---|---|---|
| GROWTH FACTORS | | | FGF family member |
| HST/KS | Transfection | | |
| INT-2 | MMTV promoter | | FGF family member |
| | Insertion | | |
| INTI/WNTI | MMTV promoter | | Factor-like |
| | Insertion | | |
| SIS | Simian sarcoma virus | | PDGF B |

RECEPTOR TYROSINE KINASES

| | | | |
|---|---|---|---|
| ERBB/HER | Avian erythroblastosis | Amplified, deleted | EGF/TGF-α/ |
| | virus; ALV promoter | Squamous cell | Amphiregulin/ |
| | insertion; amplified | Cancer; glioblastoma | Hetacellulin receptor |
| | human tumors | | |
| ERBB-2/NEU/HER-2 | Transfected from rat | Amplified breast, | Regulated by NDF/ |
| | Glioblastomas | Ovarian, gastric cancers | Heregulin and EGF- |
| | | | Related factors |
| FMS | SM feline sarcoma virus | | CSF-1 receptor |
| KIT | HZ feline sarcoma virus | | MGF/Steel receptor |
| | | | Hematopoieis |
| TRK | Transfection from | | NGF (nerve growth |
| | human colon cancer | | Factor) receptor |
| MET | Transfection from | | Scatter factor/HGF |
| | human osteosarcoma | | Receptor |
| RET | Translocations and point | Sporadic thyroid cancer; | Orphan receptor Tyr |
| | mutations | Familial medullary | Kinase |
| | | Thyroid cancer; | |
| | | Multiple endocrine | |
| | | Neoplasias 2A and 2B | |
| ROS | URII avian sarcoma | | Orphan receptor Tyr |
| | Virus | | Kinase |
| PDGF receptor | Translocation | Chronic | TEL(ETS-like |
| | | Myelomonocytic | transcription factor)/ |
| | | Leukemia | PDGF receptor gene |
| | | | Fusion |
| TGF-β receptor | | Colon carcinoma | |
| | | Mismatch mutation | |
| | | Target | |

NONRECEPTOR TYROSINE KINASES

| | | | |
|---|---|---|---|
| ABI. | Abelson Mul.V | Chronic myelogenous | Interact with RB, RNA |
| | | Leukemia translocation | Polymerase, CRK, CBL |
| | | with BCR | |
| FPS/FES | Avian Fujinami SV; GA | | |
| | FeSV | | |
| LCK | Mul.V (murine leukemia | | Src family; T cell |
| | virus) promoter | | signaling; interacts |
| | insertion | | CD4/CD8 T cells |
| SRC | Avian Rous sarcoma | | Membrane-associated yr |
| | Virus | | kinase with signaling |
| | | | function; activated by |
| | | | receptor kinases |
| YES | Avian Y73 virus | | Src family; signaling |

SER/THR PROTEIN KINASES

| | | |
|---|---|---|
| AKT | AKT8 murine retrovirus | Regulated by PI(3)K?; |
| | | regulate 70-kd S6 k? |
| MOS | Maloney murine SV | GVBD; cystostatic |
| | | factor; MAP kinase |
| | | kinase |
| PIM-1 | Promoter insertion | |
| | Mouse | |
| RAF/MIL | 3611 murine SV; MH2 | Signaling in RAS |
| | avian SV | Pathway |

MISCELLANEOUS CELL SURFACE

| | | | |
|---|---|---|---|
| APC | Tumor suppressor | Colon cancer | Interacts with catenins |
| DCC | Tumor suppressor | Colon cancer | CAM domains |
| E-cadherin | Candidate tumor | Breast cancer | Extracellular homotypic |
| | Suppressor | | binding; intracellular |
| | | | interacts with catenins |
| PTC/NBCCS | Tumor suppressor and | Nevoid basal cell cancer | 12 transmembrane |
| | Drosophilia homology | Syndrome (Gorline | domain; signals |
| | | Syndrome) | through Gli homogue |
| | | | CI to antagonize |
| | | | hedgehog pathway |
| TAN-1 Notch | Translocation | T-ALI. | Signaling |
| homologue | | | |

MISCELLANEOUS SIGNALING

| | | | |
|---|---|---|---|
| BCL-2 | Translocation | B-cell lymphoma | Apoptosis |
| CBL | Mu Cas NS-1 V | | Tyrosine- |
| | | | Phosphorylated RING |
| | | | finger interact Ab1 |
| CRK | CT1010 ASV | | Adapted SH2/SH3 |
| | | | interact Ab1 |
| DPC4 | Tumor suppressor | Pancreatic cancer | TGF-β-related signaling |
| | | | Pathway |
| MAS | Transfection and | | Possible angiotensin |
| | Tumorigenicity | | Receptor |
| NCK | | | Adaptor SH2/SH3 |

GUANINE NUCLEOTIDE EXCHANGERS AND BINDING PROTEINS

| | | | |
|---|---|---|---|
| BCR | | Translocated with ABL | Exchanger; protein |
| | | in CML | Kinase |
| DBL | Transfection | | Exchanger |
| GSP | | | |
| NF-I | Hereditary tumor | Tumor suppressor | RAS GAP |
| | Suppressor | Neurofibromatosis | |
| OST | Transfection | | Exchanger |
| Harvey-Kirsten, | HaRat SV; Ki RaSV; | Point mutations in many | Signal cascade |
| N-RAS | Balb-MoMuSV; | Human tumors | |
| | Transfection | | |
| VAV | Transfection | | S112/S113; exchanger |

NUCLEAR PROTEINS AND TRANSCRIPTION FACTORS

| | | | |
|---|---|---|---|
| BRCA1 | Heritable suppressor | Mammary | Localization unsettled |
| | | Cancer/ovarian cancer | |
| BRCA2 | Heritable suppressor | Mammary cancer | Function unknown |
| ERBA | Avian erythroblastosis | | thyroid hormone |
| | Virus | | receptor (transcription) |
| ETS | Avian E26 virus | | DNA binding |
| EVII | MuLV promotor | AML | Transcription factor |
| | Insertion | | |
| FOS | FBI/FBR murine | | 1 transcription factor |
| | osteosarcoma viruses | | with c-JUN |
| GLI | Amplified glioma | Glioma | Zinc finger; cubitus |
| | | | interruptus homologue |
| | | | is in hedgehog |
| | | | signaling pathway; |
| | | | inhibitory link PTC |
| | | | and hedgehog |
| HMGI/LIM | Translocation t (3:12) | Lipoma | Gene fusions high |
| | t (12:15) | | mobility group |
| | | | HMGI-C (XT-hook) |
| | | | and transcription factor |
| | | | LIM or acidic domain |
| JUN | ASV-17 | | Transcription factor |
| | | | AP-1 with FOS |
| MLL/VHRX + | Translocation/fusion | Acute myeloid leukemia | Gene fusion of DNA- |
| ELI/MEN | ELL with MLL | | binding and methyl |
| | Trithorax-like gene | | transferase MLL with |
| | | | ELI RNA pol II |
| | | | elongation factor |
| MYB | Avian myeloblastosis | | DNA binding |
| | Virus | | |
| MYC | Avian MC29; | Burkitt's lymphoma | DNA binding with |
| | Translocation B-cell | | MAX partner; cyclin |
| | Lymphomas; promoter | | regulation; interact RB?; |
| | Insertion avianleukosis | | regulate apoptosis? |
| | Virus | | |
| N-MYC | Amplified | Neuroblastoma | |
| L-MYC | | Lung cancer | |
| REL | Avian | | NF-κB family |
| | Retriculoendotheliosis | | transcription factor |
| | Virus | | |
| SKI | Avian SKV770 | | Transcription factor |
| | Retrovirus | | |
| VHL | Heritable suppressor | Von Hippel-Landan | Negative regulator or |
| | | Syndrome | elongin; transcriptional |
| | | | elongation complex |
| WT-1 | | Wilm's tumor | Transcription factor |

CELL CYCLE/DNA DAMAGE RESPONSE

| | | | |
|---|---|---|---|
| ATM | Hereditary disorder | Ataxia-telangiectasia | Protein/lipid kinase |
| | | | homology; DNA |
| | | | damage response |
| | | | upstream in P53 |
| | | | pathway |
| BCL-2 | Translocation | Follicular lymphoma | Apoptosis |
| FACC | Point mutation | Fanconi's anemia group | |
| | | C (predisposition | |
| | | Leukemia | |
| MDA-7 | Fragile site 3 p 14.2 | Lung carcinoma | Histidine triad-related |
| | | | diadenosine 5′, 3″″- |
| | | | tetraphosphate |
| | | | asymmetric hydrolase |
| HMLI/MutL | | HNPCC | Mismatch repair; MutL |
| | | | Homologue |
| HMSH2/MutS | | HNPCC | Mismatch repair; MutS |
| | | | Homologue |
| HPMS1 | | HNPCC | Mismatch repair; MutL |
| | | | Homologue |
| HPMS2 | | HNPCC | Mismatch repair; MutL |
| | | | Homologue |
| INK4/MTS1 | Adjacent INK-4B at | Candidate MTS1 | p16 CDK inhibitor |
| | 9 p 21; CDK complexes | Suppressor and MLM | |
| | | Melanoma gene | |
| INK4B/MTS2 | | Candidate suppressor | p15 CDK inhibitor |
| MDM-2 | Amplified | Sarcoma | Negative regulator p53 |
| p53 | Association with SV40 | Mutated >50% human | Transcription factor; |
| | T antigen | Tumors, including | checkpoint control; |
| | | Hereditary Li-Fraumeni | apoptosis |
| | | Syndrome | |
| PRAD1/BCL1 | Translocation with | Parathyroid adenoma; | Cyclin D |
| | Parathyroid hormone | B-CLL | |
| | or IgG | | |
| RB | Hereditary | Retinoblastoma; | Interact cyclin/cdk; |
| | Retinoblastoma; | Osteosarcoma; breast | regulate E2F |
| | Association with many | Cancer; other sporadic | transcription factor |
| | DNA virus tumor | Cancers | |
| | Antigens | | |
| XPA | | Xeroderma | Excision repair; photo- |
| | | Pigmentosum; skin | product recognition; |
| | | Cancer predisposition | zinc finger |

5. Other Agents

It is contemplated that other agents may be used in combination with the present invention to improve the therapeutic efficacy of treatment. One form of therapy for use in conjunction with chemotherapy includes hyperthermia, which is a procedure in which a patient's tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

Hormonal therapy may also be used in conjunction with the present invention. The use of hormones may be employed in the treatment of certain cancers such as breast, prostate, ovarian, or cervical cancer to lower the level or block the effects of certain hormones such as testosterone or estrogen and this often reduces the risk of metastases.

Adjuvant therapy may also be used in conjunction with the present invention. The use of adjuvants or immunomodulatory agents include, but are not limited to tumor necrosis factor; interferon alpha, beta, and gamma; IL-2 and other cytokines; F42K and other cytokine analogs; or MIP-1, MIP-1beta, MCP-1, RANTES, and other chemokines.

6. Vaccines

It is contemplated that vaccines that are used to treat cancer may be used in combination with the present invention to improve the therapeutic efficacy of the treatment. Such vaccines include peptide vaccines or dendritic cell vaccines. Peptide vaccines may include any tumor-specific antigen that is recognized by cytolytic T lymphocytes. Yet further, one skilled in the art realizes that dendritic cell vaccination comprises dendritic cells that are pulsed with a peptide or antigen and the pulsed dendritic cells are administered to the patient.

Examples of tumor-specific antigens that are being used as vaccines in melanoma include, but are not limited to gp100 or MAGE-3. These antigens are being administered as peptide vaccines and/or as dendritic cell vaccines.

H. Pharmaceutical Formulations and Delivery

The pharmaceutical or antibody compositions disclosed herein may be administered parenterally, intravenously, intradermally, intramuscularly, transdermally or even intraperitoneally as described in U.S. Pat. No. 5,543,158; U.S. Pat. No. 5,641,515 and U.S. Pat. No. 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intratumoral and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug release capsules and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” or “pharmacologically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared.

### I. EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Cell Lines and Animals

Human melanoma cell lines A375, A375SM, TXM-13, SB-2 and WM2664 were used. Human bladder cancer cell lines 253J, 253JB-V, 253J Neo were used. Male athymic BALB/C nude mice were housed in laminar flow cabinets under specific pathogen-free conditions and were used at 8 weeks of age.

### Example 2

### Western Blot Analysis

Cell lines were seeded at 1×10⁶ in 100 mm tissue culture plates in 10 ml CMEM. After overnight incubation, the plates were washed two times in PBS and scraped off in 400 μl Triton lysis buffer, 1 μl DTT, and 4 μl protease inhibitor cocktail. After ½ h incubation on ice, the cells were centrifuged at 15,000 rpm for 15 min. The protein concentration was determined (Biorad & BSA standards), and 40 μg of protein was loaded onto a 10% SDS-PAGE and electrophoretically transferred to a 45-μm nitrocellulose membrane (Millipore, Bedford, Mass.). The membrane was blocked with 5% milk in TTBS for 1 h. Primary incubation of both cell lines was accomplished by cutting the membranes and incubating them in 1 ml of either control IgG (1:500 dilution) or anti-MUC18 or anit-IL-8 overnight. Membranes were probed with secondary antibody peroxidase-conjugated AffiniPure rabbit anti-human IgG (H+L), for 1 h and then washed with TTBS. Probed proteins were detected by ECL (Amersham Corp.) following manufacturer's protocol

### Example 3

### In Situ Fluorescent Tunel Assay

Tissues were fixed in 10% buffered formalin solution and then embedded in paraffin. Thin sections (4 μm) were prepared, and the TUNEL assay was performed using a commercial kit according to the manufacturer's protocol (Promega, Madison, Wis.). Briefly, tissue sections were deparaffinized and fixed at room temperature for 5 min in 4% paraformaldehyde. Cells were stripped of proteins by incubation for 10 min with 20 μg/ml proteinase. The tissue sections were then permeabilized by incubating them with 0.5% Triton X-100 in PBS for 5 min at room temperature. After being rinsed twice with PBS for 5 min, the slides were incubated with terminal deoxynucleotidyl transferase buffer for 10 min. Terminal deoxynucleotidyl transferase and buffer were then added to the tissue sections and incubated in a humid atmosphere at 37° C. for 1 h. The slides were washed 3 times with PBS for 5 min. Prolong solution (Molecular Probes, Eugene, Oreg.) was used to mount the coverslips. Immunofluorescence microscopy was performed using an ×40 objective on an epifluorescence microscope equipped with narrow bandpass excitation filters mounted on a filter wheel (Lud1 Electronic Products, Hawthrone, N.Y.) to select for green fluorescence. Images were captured using a cooled CCD camera (Photometrics, Tucson, Ariz.) and SmartCapture software (Digital Scientific, Cambridge, England) on a Macintosh computer. Images were further processed using Adobe PhotoShop software (Adobe Systems, Mountain View, Calif).

### Example 4

### Effect of Cell Proliferation In Vitro

Melanoma tumor cells (A375SM, TXM-13 and WM2664) were plated at a density of 3×10⁴ cells in 96-well plates. The cells were treated with 100 μg/ml of ABX-IL8 or normal IgG for 1-4 days. Cell number was determined by the dimethylthiazole dephenyl tetrazolium bromide (MTT) assay.

Briefly, after incubation for 2 h in medium containing MTT at 0.42 mg/mL, the medium was removed, and the cells were lysed in dimethyl sulfoxide. The conversion of MTT to formazan by metabolically viable cells was monitored by a 96-well microtiter plate reader at 570 nm.

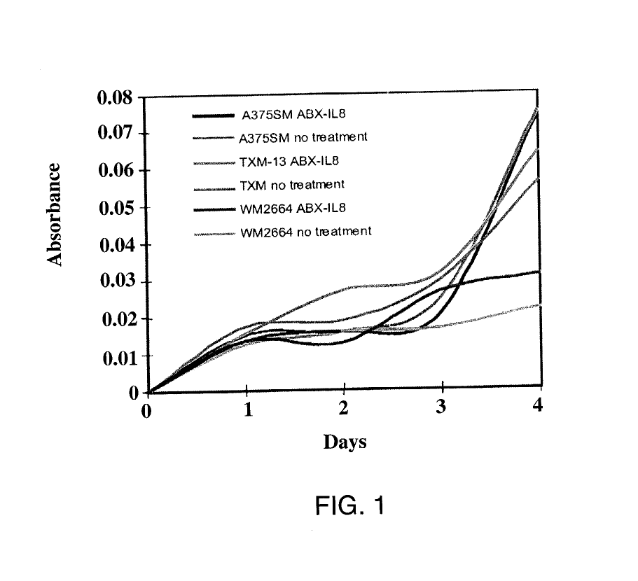

FIG. 1 shows that ABX-IL8 had no effect on proliferation of melanoma cells in vitro.

### Example 5

### IL-8 Expression in Melanoma Cells

Melanoma tumor cells (A375, A375M and SB-02) were treated with IL-8 or IgG. Protein levels in culture supernatants were determined by using a quantitative immunometric sandwich enzyme immunoassay (ELISA) kit. Absorbance of the samples was compared with the standard curve.

Treatment of A375, A375SM and SB-2 cells with 50 ug/ml of ABX-IL8 resulted in significant reduction of IL-8 in the supernatants of A375 and A375SM cells. For example, ABX-IL8 treatment resulted in a reduction of about 3000 pg/ml of IL8 in the supernatants of A375SM cells without ABX-IL8 treatment to about 900 pg/ml with ABX-IL8 tumorigenic and non-metastatic in nude mice. SB-Z melanoma cells, which do not secrete IL-8, were used as a negative control.

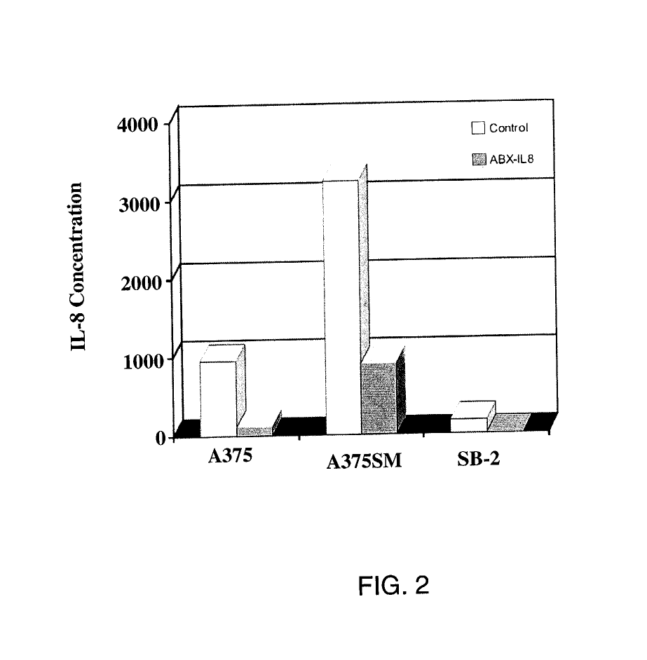

Thus, as shown in FIG. 2, ABX-IL8 neutralized IL-8 secretion by melanoma cells.

### Example 6

### Effect of ABX-IL-8 on Tumor Growth and Metastasis

For in vivo experiments, tumor cells in their exponential growth phase were harvested and resuspended in Hanks' balanced salt solution (HBSS), and diluted to the desired cell number/inoculum. To produce tumors, 5×10⁵ cells were injected s.c. in the flanks of nude mice. Mice were treated with ABX-IL8 (1 mg/3 times weekly, i.p.) or normal IgG. Tumor take and size were monitored three times per week.

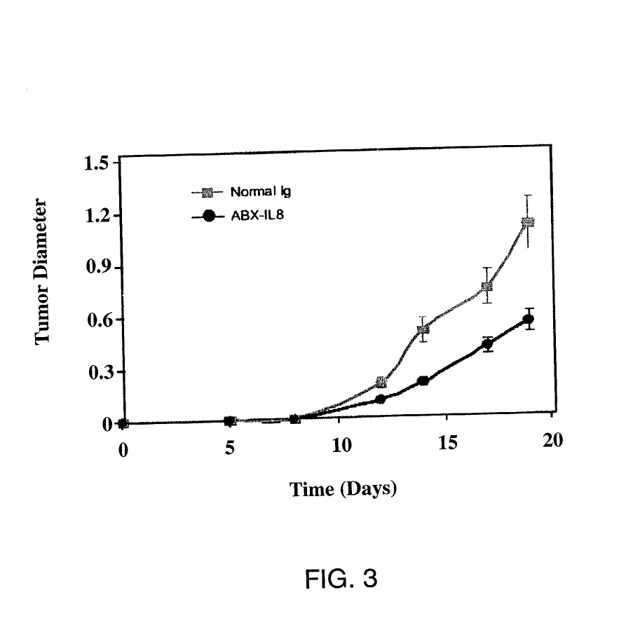

ABX-IL8 caused a significant reduction in A375SM tumor growth, as shown in FIG. 3. While the tumors in the control group reached the size of 1.2 cm in mean diameter after 20 days, the size of the tumors in the ABX-IL8 treated mice reached the size of only 0.5 cm in mean diameter after 20 days.

ABX-IL8 also caused a significant reduction of experimental lung metastases of A375SM cells in nude mice. Nude mice were injected with a total of 5×10⁵ A375SM cells intravenously (into the lateral vein) and treated with ABX-IL8 (1 mg/3 times weekly, i.p.) or normal IgG. Lung metastases were counted 60 days later. As shown in Table 2 of control IgG treated animals formed metastases in 10 of 10 mice with a median of 55 colonies (range: 30-65). In contrast, the ABX-IL8 treated mice formed metastases in 8 of 10 mice with a median of 21 nodules (range: 0-48). Median number of lung colonies were counted with the aid of a dissecting microscope.

TABLE 2
Experiment Lung Metastases of A375SM Cells
Following Treatment with ABX-IL8

Metastases^{a}

| Treatment | Median | Range | Incidence^{b} |
|---|---|---|---|
| Control | 55 | 30-65 | 10/10 |
| ABX-IL8 | 21 | 0-48 | 8/10 |

^{a}A total of 5 × 10⁵ A375SM cells were injected i.v. into nude mice. Groups of mice were treated with ABX-IL8 (1 mg/3 times weekly e.p.) or normal IgG. Lung metastases were counted 60 days later.
^{b}Number of positive mice per number injected of injected mice.

### Example 7

### Inhibition of Angiogenesis and Increase Apoptosis

Subcutaneous tumors harvested at autopsy were processed for immunostaining using rabbit polyclonal anti-CD31/PECAM-1 antibody for endothelial cells. Blood vessels were counted in each field of each sample. Apoptosis in tumor samples was analyzed by TUNEL assay.

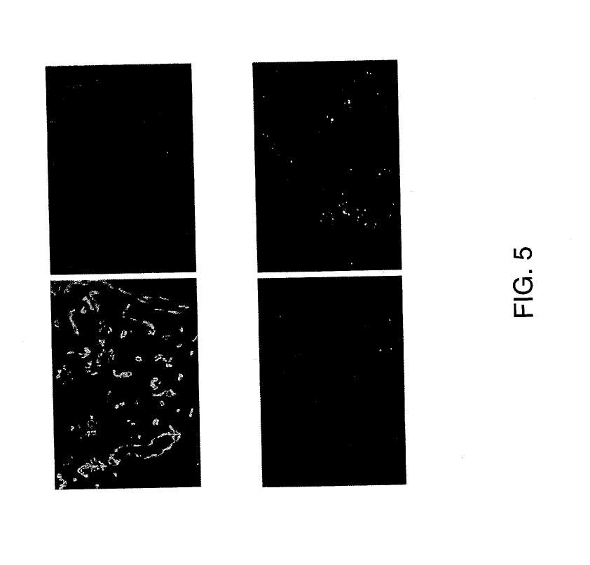

As shown in FIG. 5, ABX-IL8 inhibited angiogenesis (CD31 labeling) and caused apoptosis (TUNEL assay, which labels fragmented DNA, and therefore indicates apoptosis) of tumor cells in vivo. Growth of the control cells (without addition of ABX-IL8) was associated with massive angiogenesis within and surrounding the tumor, with very little if any tumor cells undergoing apoptosis. In contrast, in the ABX-IL8 treated mice, there was significant reduction in the number of blood vessels within and surrounding the tumors as compared to the control cells and the majority of the tumor cells underwent apoptosis.

### Example 8

### Effect of ABX-IL-8 on MMP-2 Activity

Metastatic melanoma cells were treated with ABX-IL8 or IgG for 5 days and analyzed for MMP-2 activity.

Briefly, cells (5×10³) were seeded in six-well plates. Cells were treated with 100 μg/ml ABX-IL8. 100 μg/ml IgG (Jackson Labs) or CMEM for 5 days. Conditions were serum-free on day 5. The supernatants were collected and separated on 10% SDS-PAGE containing 1 mg/ml gelatin by electrophoresis. After electrophoresis, the gel was washed at room temperature for 30 min in wash buffer (50 mM Tris-Cl, pH 7.5, 15 mM CaCl₂, 1 μM ZnCl₂, 2.5% Triton X-100) and incubated for 4 h at 37° C. in the same buffer using 1% Triton X-100. The gel was stained with a solution of 0.1% Coomassie brilliant blue R-250.

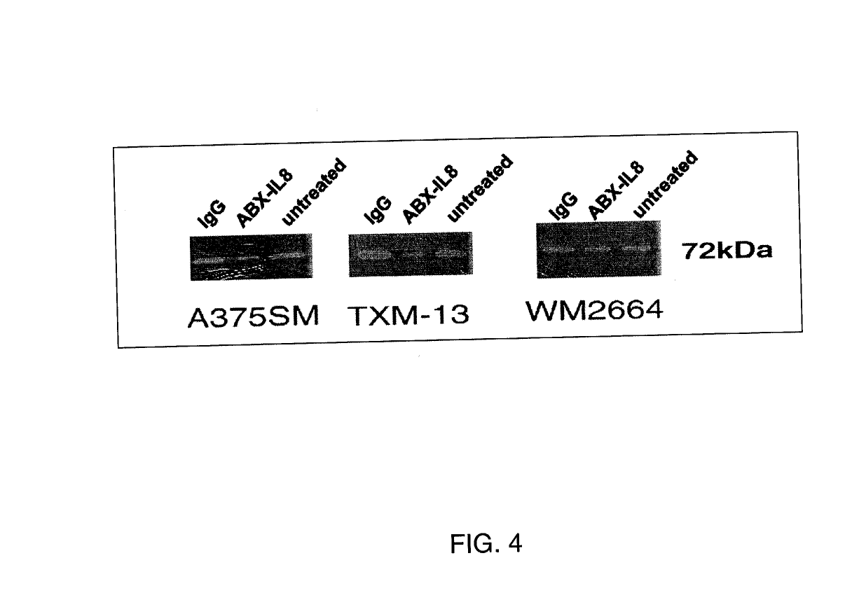

As shown in FIG. 4, ABX-IL8 suppressed MMP-2 activity in melanoma cells.

### Example 9

### ABX-IL-8 Inhibits Cell Migration

ABX-IL8 treated and untreated melanoma cells were assayed for their potential to penetrate through matrigel coated filters.

Briefly, cells (5×10³) were seeded in six-well plates. Cells were treated with 100 μg/ml IgG (Jackson Labs), 100 μg/ml ABX-IL8, or CMEM for 5 days. The cells ere exposed to Trypsin-EDTA, counted and centrifuged. 2.5×10³ cells in 500 μl of 100 μg/ml IgG (Jackson Labs), 100 μg/ml ABX-IL8, or serum-free media were placed in Matrigel invasion chambers (Beckett-Dickenson) and incubated at 37° C. for 22 hours. Conditioned CMEM was placed in the lower well of the Matrigel chamber to act as a chemoattractant. Melanoma cells were placed in the upper chamber. After 22 hours of incubation, the cells of the lower surfaces of the filter were strained and counted.

As shown in Table 3, ABX-IL8 inhibited migration of melanoma cells through matrigel filters.

ABX-IL8 treated and untreated melanoma cells were assayed for their potential to penetrate through matrigel coated filters. Melanoma cells (2×10⁵) were placed in the upper chamber. After 22 hours of incubation, the cells on the lower surface of the filter were stained and counted.

TABLE 3
ABX-IL8 Inhibits Migration of Melanoma Cells Through Matrigel Filters

| Cell Lines | Treatment | Migrated Cells + SEM |
|---|---|---|
| TXM-13 | IgG | 184 ± 3 (p < 0.05) |
| TXM-13 | ABX-IL8 | 45 ± 10 |
| TXM-13 | None | 163 ± 15 |
| A375SM | IgG | 125 ± 43 |
| A375SM | ABX-IL8 | 12 ± 1 |
| A375SM | None | 435 ± 33 (p < 0.001) |

ABX-IL8 treated and untreated melanoma cells were assayed for their potential to penetrate through matrigel coated filters. Melanoma cells (2 × 10⁵) were placed in the upper chamber. After 22 hours of incubation, the cells on the lower surface of the filter were stained and counted.

### Example 10

### Effect of ABX-IL8 Cell Proliferation

253J B-V bladder cancer cells were treated with different doses of ABX-IL8 and IgG. Cell number was determined by the dimethylthiazole dephenyl tetrazolium bromide (MTT) assay.

Briefly, after incubation for 2 h in medium containing MTT at 0.42 mg/mL, the medium was removed, and the cells were lysed in dimethyl sulfoxide. The conversion of MTT to formazan by metabolically viable cells was monitored by a 96-well microtiter plate reader at 570 nm.

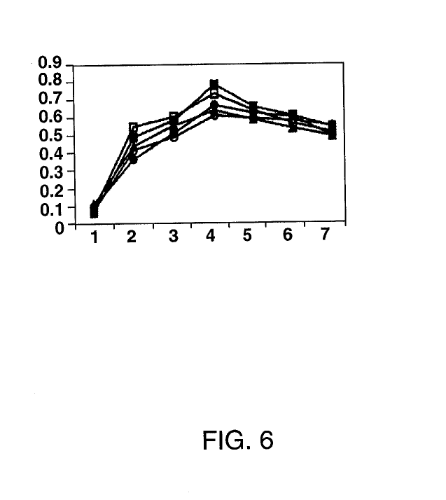

FIG. 6 shows that this was no significant effect on the proliferation rate of the cells after 7 days of treatment. Thus, ABX-IL8 has no direct cytotoxic effects on the cells.

### Example 11

### II-8 Expression and Neutralization

Poorly tumorigenic bladder cancer cells, (253J), were transfected with pcDNA-sense-IL-8. Protein levels in culture supernatants were determined by using a quantitative immunometric sandwich enzyme immunoassay (ELISA) kit. Absorbance of the samples was compared with the standard curve.

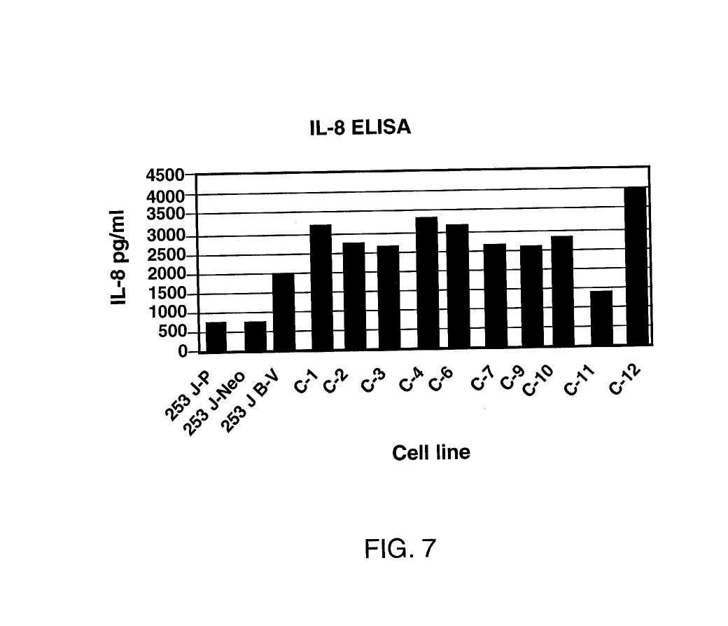

FIG. 7 shows that the poorly tumorigenic bladder cancer cells that were transfected with pcDNA-sense-IL-8 resulted in a significant increase in the expression of IL-8 that was comparable to the highly tumorigenic bladder cancer cells (253J B-V).

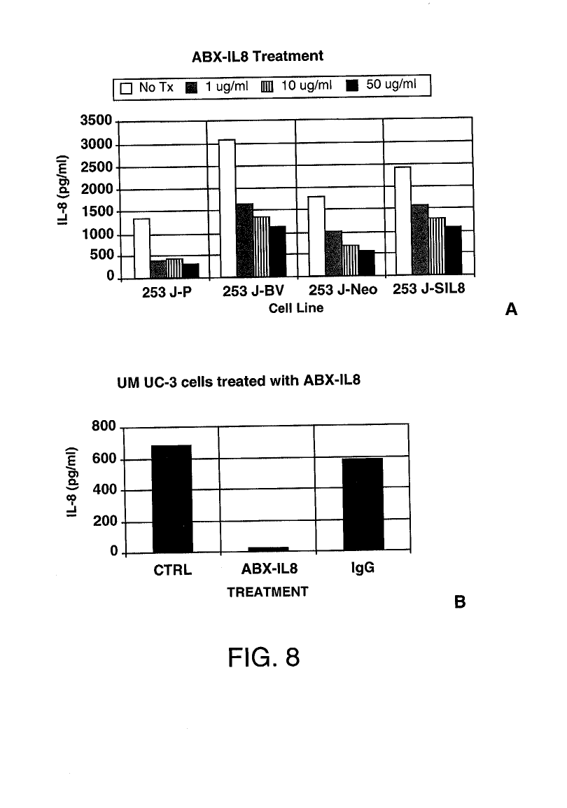

Next, to determine the neutralization effect of ABX-IL-8, cells were treated with increasing doses of ABX-IL8 or control IgG. FIG. 8A and FIG. 8B show a dose-dependent decrease in the level of IL-8 in different bladder cancer cell lines, but not with IgG.

### Example 12

### Collagenase Activity and Transcription

Cells (5×10³) were seeded in six-well plates. 253J, 253J B-V, 253J-Neo and 253J-sense-IL8 transfected cells were treated with ABX-IL8 or IgG for 5 days. Conditions were serum-free on day 5. The supernatants were collected and separated on 10% SDS-PAGE containing 1 mg/ml gelatin by electrophoresis. After electrophoresis, the gel was washed at room. temperature for 30 min in wash buffer (50 mM Tris-Cl, pH 7.5, 15 mM CaCl₂, 1 μM ZnCl₂, 2.5% Triton X-100) and incubated for 4 h at 37° C. in the same buffer using 1% Triton X-100. The gel was stained with a solution of 0.1% Coomassie brilliant blue R-250.

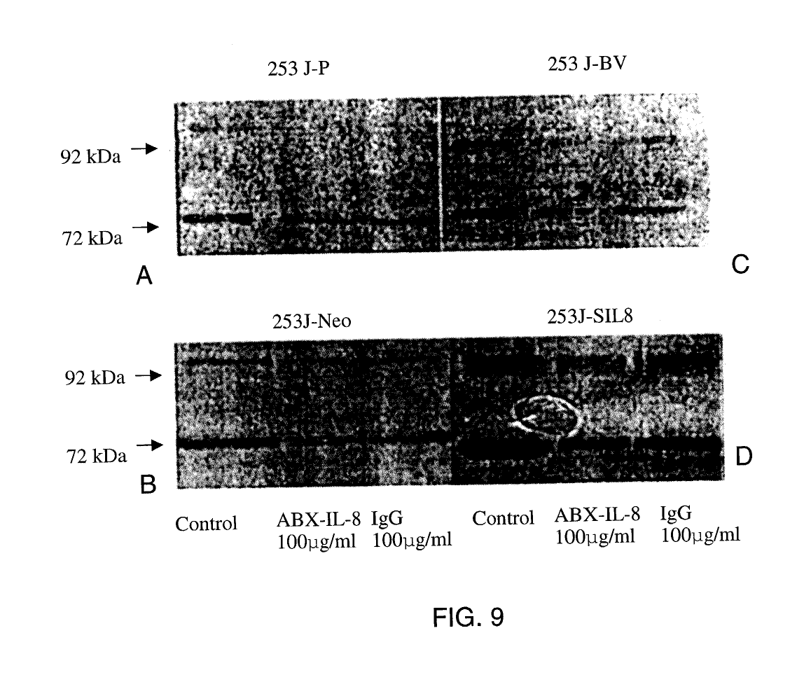

As shown in FIG. 9A, FIG. 9B. FIG. 9C and FIG. 9D, there was a significant decrease in MMP-9 and MMP-2 activity.

### Example 13

### Inhibitation of Cell Migration

Next, bladder cancer cells were treated with ABX-IL8 or IgG to determine whether a decrease in collagenase activity is accompanied by a decrease in the invasive potential of bladder cancer cells.

Briefly, cells (5×10³) were seeded in six-well plates. Cells were treated with 100 μg/ml IgG (Jackson Labs), 100 μg/ml ABX-IL8, or CMEM for 5 days. The cells ere exposed to Trypsin-EDTA, counted and centrifuges. 2.5×10³ cells in 500 μl of 100 μg/ml IgG (Jackson Labs), 100 μg/ml ABX-IL8, or serum-free media were placed in Matrigel invasion chambers (Beckett-Dickenson) and incubated at 37° C. for 22 hours. Conditioned CMEM was placed in the lower well of the Matrigel chamber to act as a chemoattractant. The cells were placed in the upper chamber. After 22 hours of incubation, the cells of the lower surfaces of the filter were strained and counted.

Table 4 illustrates that there was significant decrease in the number of cells invading through a Matrigel-coated membrane after treatment with ABX-IL8.

TABLE 4

| | 253J B-V | 253J B-V |
|---|---|---|
| Control | 125 ± 44 | 113 ± 43 |
| ABX-IL8 (100 μg/ml) | 26 ± 9 (p < 0.008) | 50 ± 20 (p < 0.032) |
| IgG (100 μg/ml) | 90 ± 50 (NS) | 97 ± 37 (NS) |

*Mean number of invading cells ± SD per membrane

### Example 14

### Effect on Transcription

In-situ hybridization was performed to determine the mRNA expression. Cells were treated in vitro and then fixed on slides for mRNA in-situ hybridization. Table 5 shows a significant downregulation of MMP-9, and MMP-2 at the level of transcription.

TABLE 5
mRNA in situ hybridization

| | |
|---|---|
| MMP-9 | MMP-2 |

| | Control | ABX-IL8 | Control | ABX-IL8 |
|---|---|---|---|---|
| 253 J B-V | 3.8 ± .77 | 2.5 ± .40* | 3.8 ± .46 | 3.0 ± .44* |
| 253 J-P | 3.8 ± .41 | 3.4 ± .49 | | |
| 253 J S-IL8 | 4.2 ± .46 | 3.8 ± .46 | | |

*All p values < 0.005
*Mean O.D. ± SD

Thus, the above data illustrate that the down regulation of MMP's activity, as seen on zymography and invasion assay, was at the level of transcription.

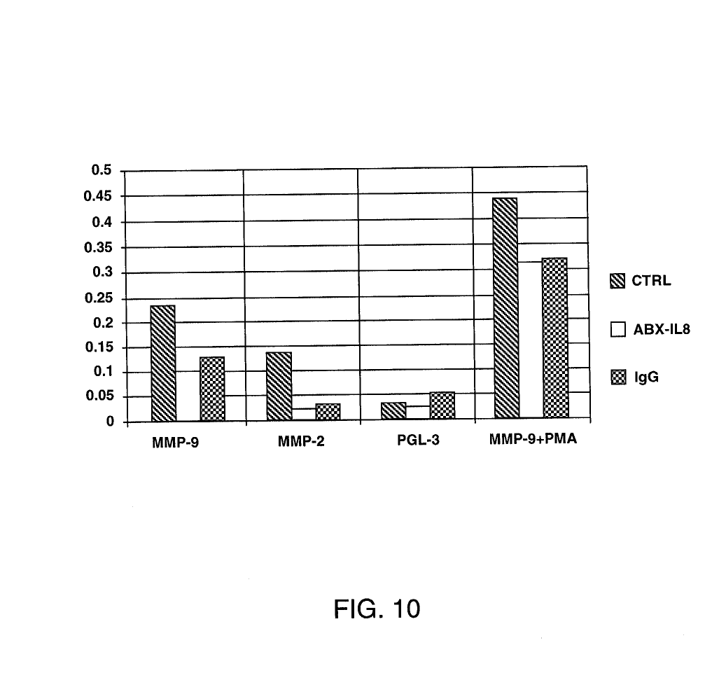

Also, a luciferase assay was preformed to determine the effect on the MMP-9 and MMP-2 promoter activity after treatment with ABX-IL8. FIG. 10 shows that there was a decrease in the MMP-9 and MMP-2 promoter activity after treatment with ABX-L8 when compared with controls or IgG treatment. This effect was seen on both constitutively expressed, inducible (with PMA) MMP-9.

Thus, ABX-IL8 mediated downregulation of collagenase activity is associated with a decreased invasive potential in these cells. The downregulation of MMP's following treatment with ABX-IL8 is at the transcription level, as evident by a decrease in mRNA expression and MMP-9/-2 promoter activity.

### Example 15

### Flow Cytofluorometry

Quantitative analysis of MUC18 on cell surfaces was determined by FACS analysis. A375SM and SB2 cells were plated in 6-well plates and incubated for 24 h. After attachment, wells were scraped with a disposable cell scraper (Sarstedt) and incubated with control IgG or ABX-MUC18 for 1 h at 4° C. After several washings with FACS buffer, all samples were incubated with R-phycoerythrin-conjugated AffiniPure F (ab′)² fragment goat anti-human IgG (H+L) 1:200 dilution (Jackson Labs, West Grove, Pa.) for 1 h at 4° C. in the dark. The cells were fixed in 1% paraformaldehyde in PBS and examined by cytofluorometry.

### Example 16

### Generation of Specific Fully Human Anti-MUC18

ABX-MUC18 is a human IgG2 monoclonal antibody directed against human MUC18/MCAM that was generated using Abgenix's propriety XenoMouse mice. A full-length protein encoded by MUC18 cDNA or small peptides corresponding to different regions of the protein were used as antigen to immunize the mice. Thirty-five hybridomas were initially generated and screened for their ability to recognize the 113 kDa MUC18 protein on Western blot and FACS analyses. Clone A 15 6.9 was chosen for further in vitro and in vivo analyses and designated as ABX-MUC18.

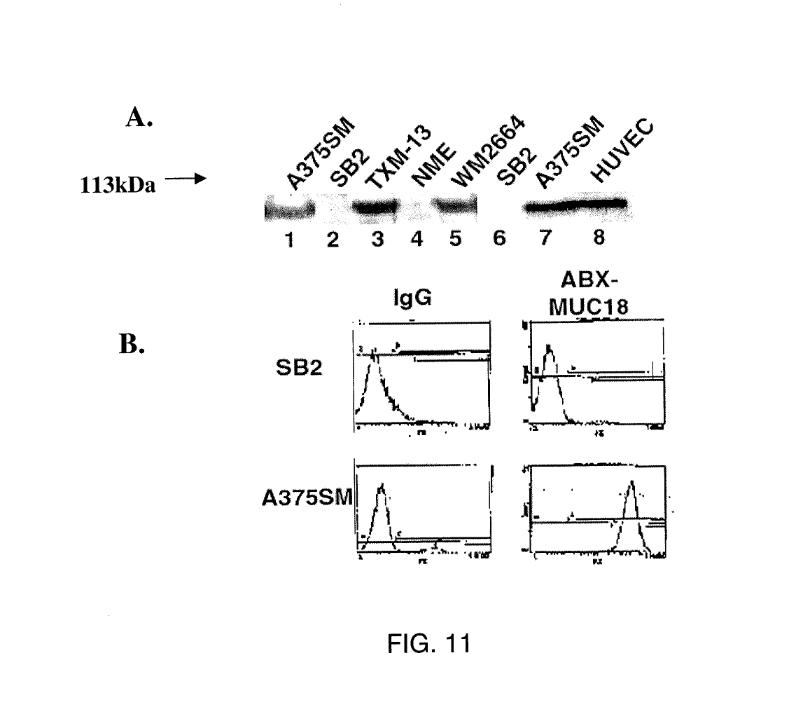

FIG. 11 shows that ABX-MUC18 recognized and detected one band of the 113-kDa MUC18 protein in the metastatic melanoma cell lines A375SM, TXM-13, and WM2664 [all expressed high levels of MUC18 (Xie, et al., 1997), FIG. 11A, lanes 1, 3, 5 and 7], but not in the nonmetastatic melanoma cell line SB-2 [MUC-18 negative (Xie, et al., 1997), FIG. 11A, lanes 2 and 6]. FIG. 11 also shows that ABX-MUC18 detected the 113-kDa protein on HUVEC (FIG. 11A, lane 8), but not on murine nude mouse endothelial cells (NME, lane 4). The MUC18 adhesion molecule was also detected with ABX-MUC18 by FACS analysis on the cell surface of A375SM cells, but not on SB-2 cells (FIG. 11B). Thus, ABX-MUC18 specifically bound and detected the 113-kDa MUC18 receptor on metastatic human melanoma and HUVEC cells.

### Example 17

### Disruption of Spheroid Formation by Metastatic Melanoma Cells

Clumping or emboli formation of tumor cells is an important step prior to extravasation and metastasis. To evaluate the role of ABX-MUC18 in homotypic aggregation, the inventor analyzed the ability of MUC18-negative (SB-2), and MUC18-positive (A375SM) cells to grow in a three-dimensional culture system, i.e., as so-called multicellular spheroids (Rofstad et al., 1986; Kobayash et al., 1993) in the presence of ABX-MUC18.

Multicellular spheroids were generated by the liquid overlay technique (Rofstad et al., 1986; Kobayash et al., 1993). Briefly, 24-well tissue culture plates (Costar) were coated with 250 μl of prewarmed 1% SeaPlaque agarose (FMC Bioproducts, Rockland, Me.) solution in serum-free MEM. After the agarose was allowed to solidify and to form a thin layer on the bottom of the dish, a single-cell suspension of A375SM or SB2 (1×10⁵) was diluted in 25 μl of hybridoma medium and plated with 475 μl of control IgG (1:200 dilution) or ABX-MUC18, and incubated at 37° C. in 5% CO₂-95% air. After 24 h spheroid formation was determined. Images were captured by bright-field microscopy and photographed in digital format.

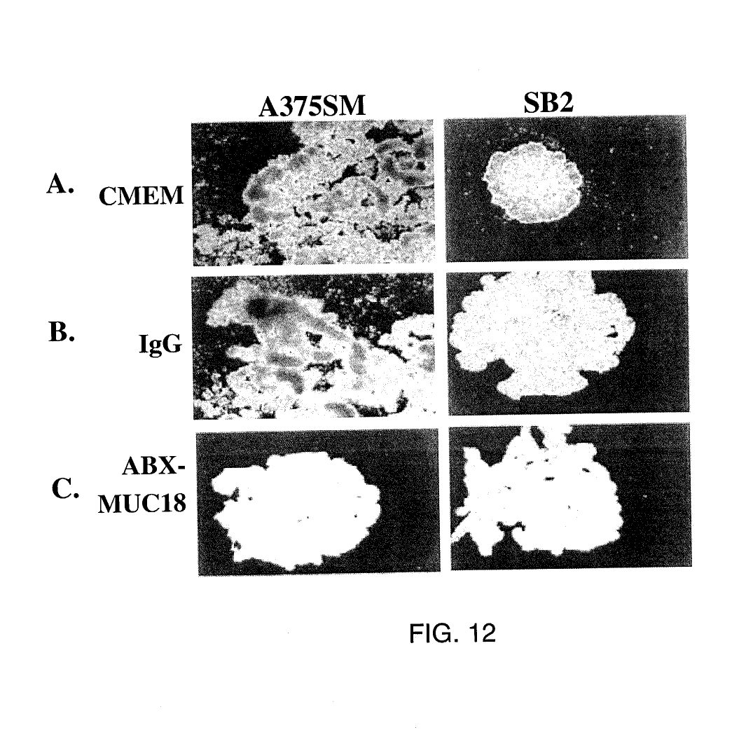

In the above culture conditions, adherence and formation of monolayers by the cells at the bottom of the dish were prevented by the presence of a thin layer of solid agarose, thus forcing the cells to from tumor-like homotypic multicellular aggregates. Under these conditions, the MUC18-negative SB-2 cells grew in monolayers whereas the MUC18-positive A375SM cells formed spheroids (FIG. 12). When the cells with ABX-MUC18 (added at time 0) were cultured, spheroid formation was disrupted only in A375SM, not SB-2, cells (FIG. 12C). This effect was not observed when control IgG antibody was added to the cultures (FIG. 12B).

Thus, this data confirms ABX-MUC18's ability to disrupt spheroid formation by metastatic MUC18-postiive melanoma cells.

### Example 18

### Suppression of Tumorigenicity of Human Melanoma Cells by ABX-MUC18

As expression of MUC18/MCAM by melanoma cells contributes to tumor growth and metastasis, the inventor determined the effect of ABX-MUX18 on tumorigenicity and metastasis of human melanoma cells in nude mice.

Briefly, MUC18-positive A375SM (1×10⁵) and WM2664 (5×10⁵) cells were injected s.c. into nude mice (n=10). Three days later, animals injected with A375SM were injected with 1 mg ABX-MUC18 i.p. once weekly; animals injected with WM2664 cells were injected with 100 μg ABX-MUC18 twice weekly for a period of 4-5 weeks. Control animals were injected with normal IgG antibody.

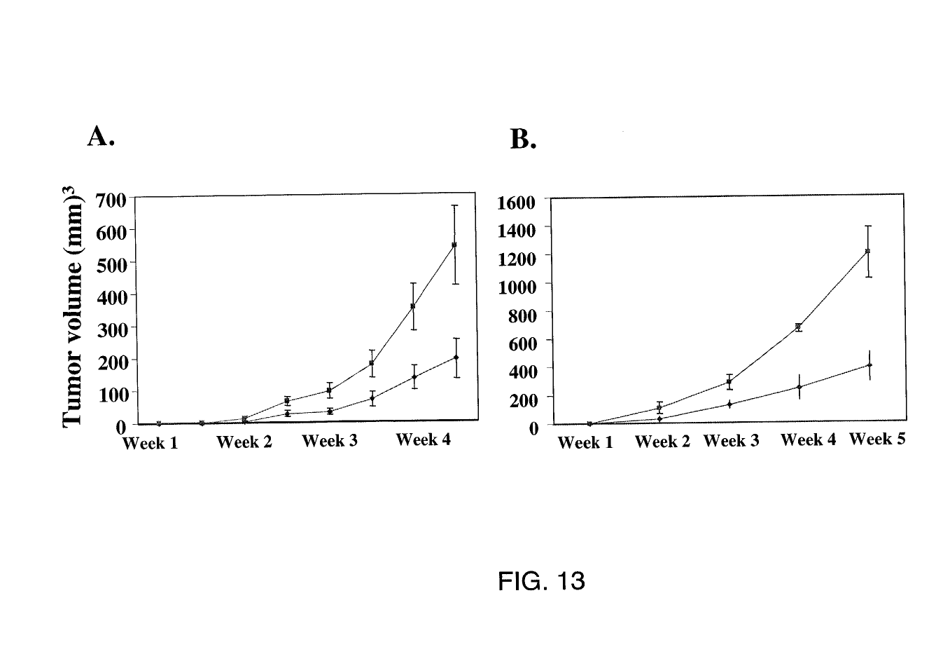

Tumor cells in the animals treated with control IgG grew progressively and produced large tumors reaching 1.2 cm in mean diameter for WM2664 and 0.5 cm for A375SM (FIGS. 13A and 13B). In contrast, treatment with ABX-MUC18 reduced tumor growth to approximately 0.4 cm in mean diameter for WM2664 and 0.18 cm for A375SM during the same periods of time (FIG. 13).

### Example 19

### Suppression of Metastasis of Human Melanoma Cells by ABX-MUC18

Next, to determine the effect of ABX-MUC18 on metastasis of human melanoma cells, A375SM and WM2664 cells were injected i.v. into nude mice to produce experimental lung metastasis. Three days later, animals injected with tumor cells were also injected with ABX-MUC18 or control IgG i.p., once or twice weekly as indicated for a period of 4 weeks for A375SM and 8 weeks for WM2664 cells. Two different doses of ABX-MUC18 (100 μg or 1 mg) were used to treat the injected animals.

As shown in Table 6, both the incidence and number of lung metastasis of both melanoma cell lines were lower in ABX-MUC18-treated mice than in the control IgG-treated group. Both doses of ABX-MUC18 were equally effective in inhibiting metastases formation. In IgG-treated mice, A375SM cells produced numerous lung metastases (median of 17, range 5-34) whereas the WM2664 cells produced a median of 11 lung colonies (range 4-21). In contrast, treatment with ABX-MUC18 significantly inhibited the ability of both A375SM and WM2664 cells to form metastasis in nude mice (median 0-2, range 0-8, p<0.001). Collectively, these data demonstrate that treatment of mice with ABX-MUC18 suppressed melanoma tumor growth and metastasis.

TABLE 6
Experimental lung metastasis of A375SM and WM2664 cells in
nude mice following treatment with ABX-MUC18

| | | | Metastasis | |
|---|---|---|---|---|
| Cell Line | Treatment | Median | Range | Incidence |
| A375SM | IgG | 17 | 5-34 | 5/5 |
| | ABX-MUC18 | 1* | 0-6 | 3/5 |
| | 100 μg/ml | | | |
| | ABX-MUC18 | 0* | 0-1 | 2/5 |
| | 1 mg/ml | | | |
| WM22664 | IgG | 11 | 4-21 | 5/5 |
| | ABX-MUC18 | 1* | 0-4 | 3/5 |
| | 100 μg/ml | | | |
| | ABX-MUC18 | 2* | 0-8 | 2/5 |
| | 1 mg/ml | | | |

*P < 0.01 as determined by Mann-Whitney U test.

### Example 20

### Cell Proliferation and ABX-MUC18

The effect of ABX-MUC18 on cell proliferation was measured. Briefly, A375SM, WM2664 melanoma, and HUVEC cells were treated with 100 μg/ml ABX-MUC18 or control IgG for 1-4 days. Cell proliferation was determined by MTT assay, which determines the relative cell numbers based on the conversion of MTT to formazan in viable cells. Briefly, MTT (40 μg/ml) was added to each well and incubated for 2 h. The medium was removed, and 100 μl of DMSO was added to lyse cells and solubilize formazan. Absorbance was determined on a microplate reader.

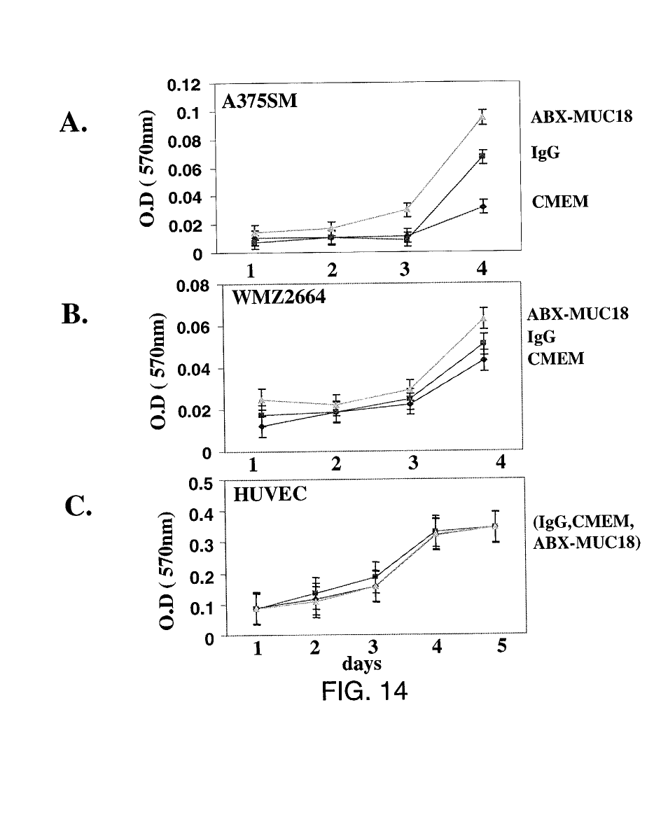

The decrease in tumor growth and in the ability to produce lung metastases attributed to ABX-MUC18 was not due to differences in cell division time, as no significant differences in cell doubling time were found when A375SM and WM2664 were cultured in vitro in the presence of ABX-MUC18 (FIGS. 14A and 14B). These data suggest that ABX-MUC18 did not have direct effect on melanoma cell proliferation. ABX-MUC18 also did not alter the proliferation rate of HUVEC in vitro (FIG. 14C).

### Example 21

### Effect of ABX-MUC18 on MMP-2 Activity

MMP-2 activity was determined on substrate impregnated gels with modifications. A375SM cells were plated and allowed to attach for 24 hours. Cells were treated with anti-MUC18 antibody (Abgenix), IgG (Jackson, Labs) or CMEM for 5 days. The supernatant was collected, adjusted for cell number, loaded, and run on 10% SDS-PAGE gels under non-reducing conditions, followed by 30 minute washings in 2.5% Triton X-100, 50 mM Tris HCL pH 7.5, 5 mM CaCl₂, 1 uM ZnCl₂ in dH₂O. The gels were then incubated for 16 hours at 37° C. in 1% Triton X-100, 50 mM Tris HCl pH7.5, 5 mM CaCl₂, 1 uM ZnCl₂ in dH₂O. The gels were stained with 5% Coomassie Blue (BioRad) in methanol/acetic acid/H₂O (30:10:60), and they were destained with methanol, acetic acid, and dH₂O, 40:10:50.

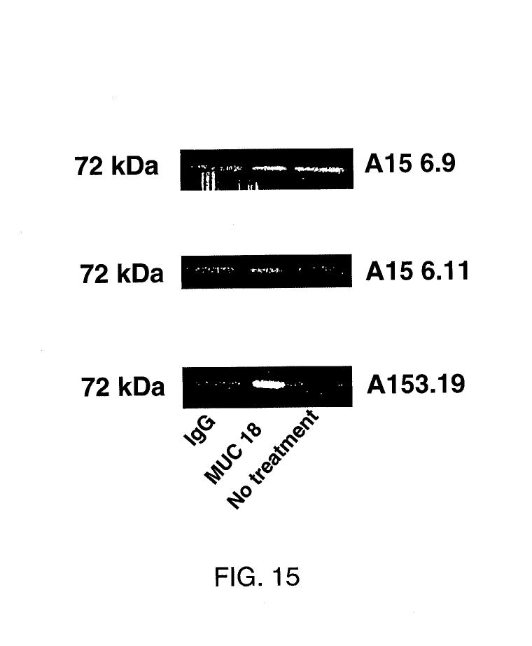

FIG. 15 shows the effect of the three anti-MUC18 antibodies A15 6.9, A15 6.11, and A15 3.19 on the activity of MMP-2 (collagenase type IV, 72 kDa). Treatment of A375SM melanoma cells (which express high levels of MCAM/MUC18) resulted in the inhibition of MMP-2 activity by 2.5-fold.

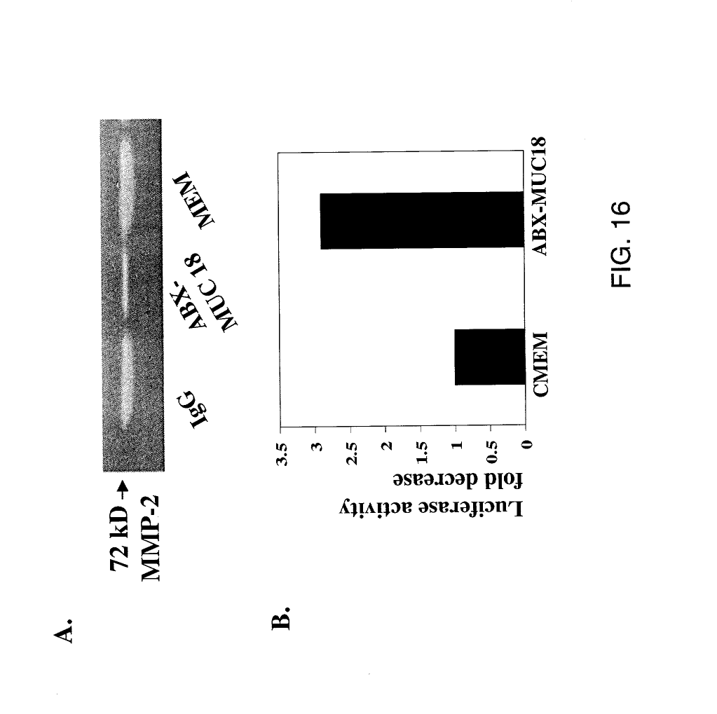

Also, as shown in FIG. 16A, the 72-kDa MMP-2 collagenase activity of ABX-MUC18-treated A375SM cells was significantly lower than that of either IgG-treated or untreated (grown in CMEM) control cells.

### Example 22

### Effect of ABX-MUC-18 MMP-2 Transcription

To examine the effect of ABX-MUC18 on MMP-2 transcription, the promoter activity of MMP-2 in ABX-MUC18-treated and control melanoma cells was analyzed.

The MMP-2 promoter construct was generated by cutting the MMP-2 promoter region, −390 to +290 (31), out of p682 basic (CAT driven MMP-2 promoter) (Luca et al., 1997) at the HindII/XbaI sites and ligating into pGEM-9Zf(−) vector (Promega) using the same sites. The MMP-2 promoter region was then removed via the SpeI/SalI sites and ligated into the pGL3-Enhancer (Promega, Madison, Wis.) (Gershenwald et al., 2001). Melanoma cells were treated with 100 μg/ml ABX-MUC18, control IgG, or CMEM for 4 days of and then transfected with 10 ng of pB-actin-RL (Huang et al., 1998) and 2 μg plasmid DNA of either luciferase basic vector, SV40 positive control, or MMP-2 promoter vector, using 10 μl Lipofectin reagent (GIBCO Life Technologies, Rockville, Md.). The medium was changed and treatments added after 12 h. Cells were lysed and analyzed using dual luciferase assay (Promega, Madison, Wis.) and Ascent Lumiskan plate reader and software (Huang et al., 1998).

Consistent with a decreased MMP-2 collagenase activity, MMP-2 promoter activity in ABX-MUC18-treated A375SM cells was decreased by X3 fold, when compared to IgG-treated and untreated cells (FIG. 16B). These results suggest that MUC18 may directly regulate MMP-2 expression at the transcriptional level and that blocking of MUC18 by ABX-MUC18 suppressed MMP-2 expression and activity in melanoma cells.

### Example 23

### Effect of ABX-MUC18 on Attachment to Cells

Attachment of A376SM cells to endothelial cells treated with 12 μg/ml (A15 6.9/A15 6.11) and 2.5 μg/ml (A15 3.19) anti-MUC18 antibody, IgG, or serum free media with aliquot of hybridoma media, were measured by plating HUVEC cells and allowing them to attached for 24 hours. A thin overlay of 2% BSA was placed in each well and incubated at 37° C. for 1 hour. A375SM cells with or without treatment were added to each well and incubated for 1 hour at 37° C. Wells were rinsed and cells in each well were counted. Results are presented as percent of cells adhered from total number of cells seeded.

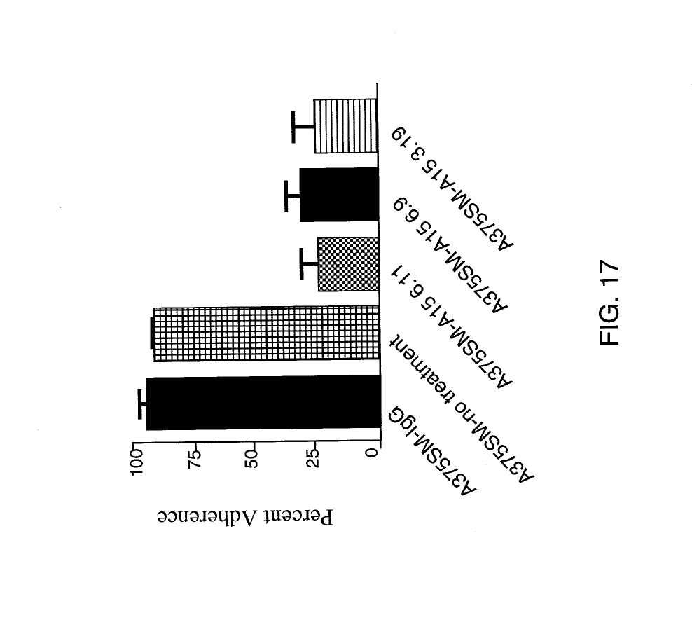

FIG. 17 shows that all three antibodies to MUC18: A15 6.11, A15 6.9 inhibited the adherence of A375SM cells to HUVEC cells. While 100 percent of three initial seeded control cells (untreated and control IgG treated) adhered to the HUVEC cells, only 25 percent of the antibody-treated cells were found attached. Thus, these antibodies disrupt the binding of melanoma cells to HUVEC cells.

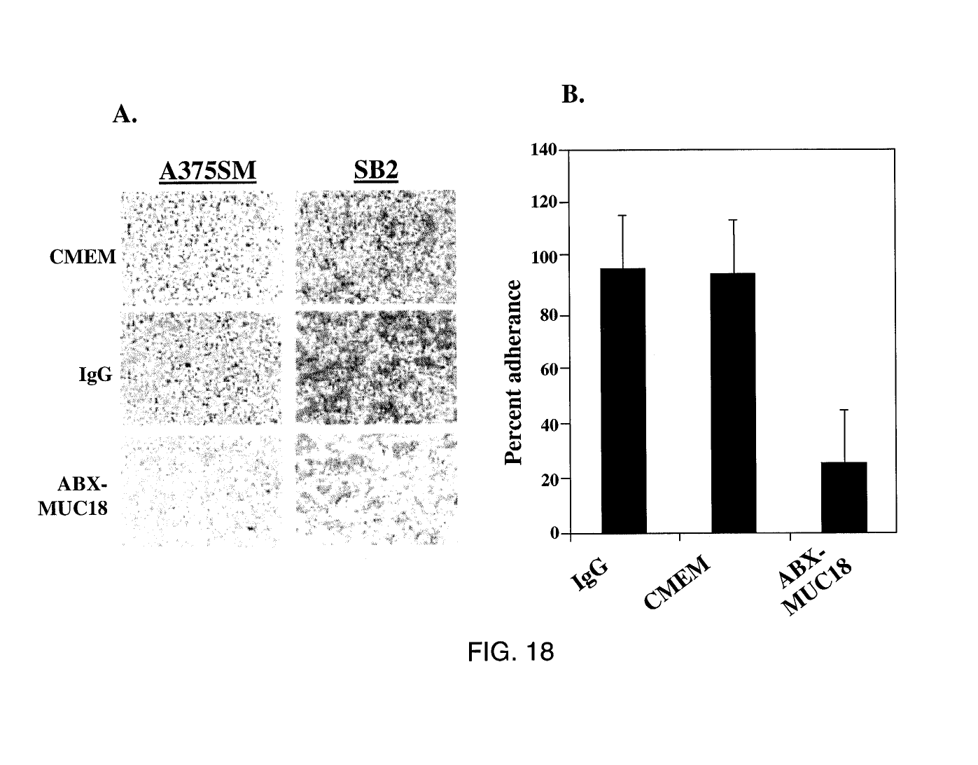

Yet further, FIG. 18 shows that very few SB-2 melanoma cells attached to HUVEC cells and that the addition of control IgG or ABX-MUC18 antibody to the cultures did not alter SB-2 HUVEC cell interactions (FIG. 18A). In contrast, A375SM cells attached to HUVEC cells and treatment with ABX-MUC18, but not with control IgG antibody, inhibited A375SM-HUVEC interaction (FIG. 18A). Quantitative summary of the data shows that (FIG. 18B) ABX-MUC18 reduced the interaction of A375SM with HUVEC by 80%. These data provide a further potential mechanism by which ABX-MUC18 may inhibit extravasation and hence metastasis.

### Example 24

### Effect of Anti-MUC18 on Migration Through Matrigel-Coated Filters

Invasion of highly metastatic A375SM or WM2664cells was measured by plating 5×10³ cells on well plates and allowing them to attach for 24 hours. After 5 days of treatment with anti-MUC18 antibody, IgG, or no treatment, the cells were released from the plates by a brief exposure to Trypsin-EDTA (Gibco-BRL), counted and centrifuged. Matrigel invasion chambers (Beckett-Dickenson) were primed according to manufacturer directions. CMEM was placed in the lower well to act as a chemoattractant. 2.5×10³ cells in 500 μl of 12 μg/ml (A15 6.11 and A15 6.9) or 2.5 μg/ml (A15 3.19) anti-MUC18 antibody, IgG (with hybridoma media aliquot), or serum free media (untreated cells with hybridoma media aliquot) were placed in the upper chamber of the Matrigel plate and incubated at 37° C. for 22 hours. Upper chamber wells were then scrubbed and stained according to manufacturer directions. The matrix was mounted on slides and cells counted. Results are presented as percent of cells migrated from total number cells seeded.

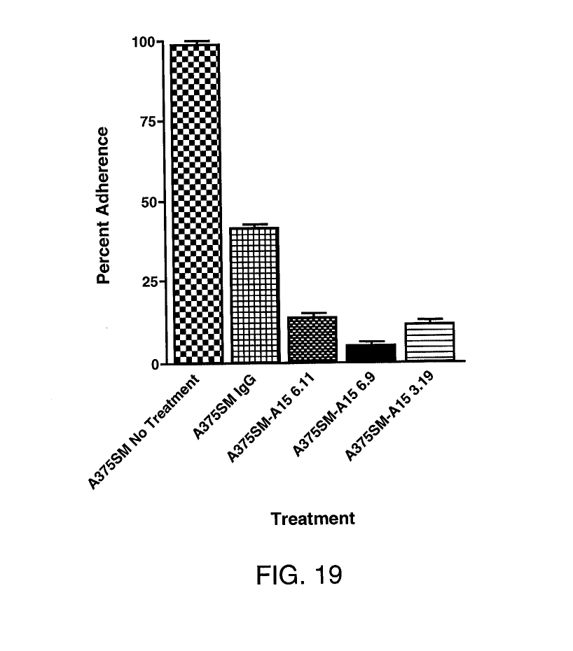

FIG. 19 shows that parental A375SM cells have a high potential to penetrate through Matrigel-coated filters, while the ability of anti-MUC18 treated cells was dramatically inhibited.

Table 7 also illustrates that A375SM and WM2664 cells treated with ABX-MUC18 exhibited a significant less invasion through Matrigel-coated filters, than IgG-treated or untreated cells (2068±129 vs. 57±8 for A375SM, p<0.001; and 1866±130 vs. 56±7, p<0.001 for WM2664).

TABLE 7
Invasion of ABX-MUC18-treated melanoma cells
through Matrigel-coated filters

| Cell Line | No. Migrated Cells ± SD |
|---|---|
| A375SM MEM | 2575 ± 94 |
| A375SM IgG | 2068 ± 129 |
| A375SM ABX-MUC18 | 57 ± 8 (p < 0.001) |
| WM2664 MEM | 1857 ± 57 |
| WM2664 IgG | 1866 ± 130 |
| WM2664 ABX-MUC18 | 56 ± 7 (p < 0.001) |

Collectively, results indicate that blockade of MUC18 in melanoma cells by ABX-MUC18 inhibited their ability to penetrate through the basement membrane. Collectively, the data indicate that inactivation of MMP-2 by ABX-MUC18 in melanoma cell may account for the decrease in their metastatic potential.

### Example 25

### Decreased Expression of MMP-2 in Tumors Treated with ABX-MUC18

To determine whether ABX-MUC18 suppressed the expression of MMP-2 in vivo, immunohistochemical (IHC) analysis on melanoma specimens derived from the implanted A375SM and WM2664 tumors was preformed.

For CD31 and MMP-2 staining, sections of frozen tissues were prepared from tumor xenografts. The slides were then rinsed twice with PBS, and endogenous peroxidase was blocked by the use of 3% hydrogen peroxide in PBS for 12 min. The samples were then washed three times with PBS and incubated for 10 min at room temperature with a protein-blocking solution consisting of PBS (pH 7.5) containing 5% normal horse serum and 1% normal goat serum. Excess blocking solution was drained, and the samples were incubated for 18 h at 40 C. with a 1:100 dilution of monoclonal rat anti-CD31 (1:100) Ab or anti-MMP-2 (1:100) (PharMingen, San Diego, Calif.). The samples were then rinsed 4 times with PBS and incubated for 60 min at room temperature with the appropriate dilution of peroxidase-conjugated antimouse IgG1antirabbit IgG, or antirat IgG. The slides were rinsed with PBS and incubated for 5 min with diaminobenzidine (Research Genetics, Huntsville, Ala.). The sections were then washed three times with distilled water and counterstained with Gill's hematoxylin. Sections (4 μm thick) of formalin-fixed, paraffin-embedded tumors were also stained with H&E for routine histological examination.

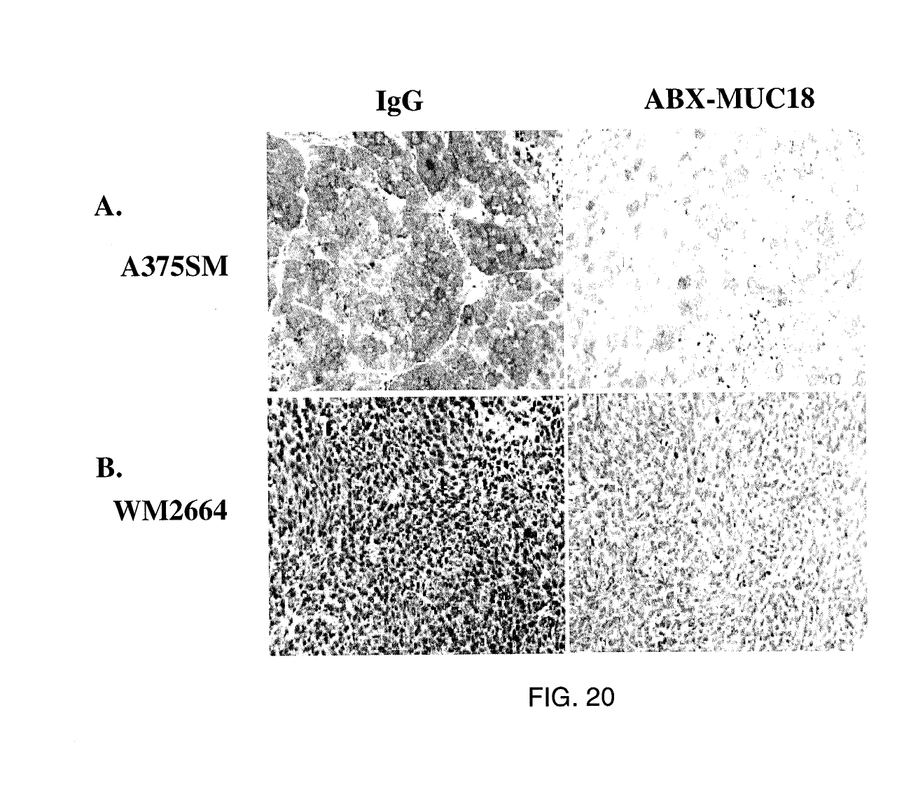

As shown in FIG. 20, MMP-2 staining was strong in IgG-treated A375SM and WM2664 tumors, but was considerably decreased in ABX-MUC18-treated tumors. Thus, blocking of MUC18 by ABX-MUC18 antibody in melanoma cells inhibited the expression of MMP-2 gene in vivo as well as in vitro.

### Example 26

### Inhibition of Tumor Angiogenesis in ABX-MUC18-Treated Cells

Tumor microvessel density (MVD) and apoptosis (TUNEL) was measured in subcutaneous melanoma xenografts.

Briefly, A375SM and WM2664 cells were injected s.c. into nude mice and treated with ABX-MUC18 or control IgG. Thirty to 60 days later, the resulting s.c. tumors with similar size were resected and processed for immunohistochemical analysis, for CD31 and TUNEL staining.

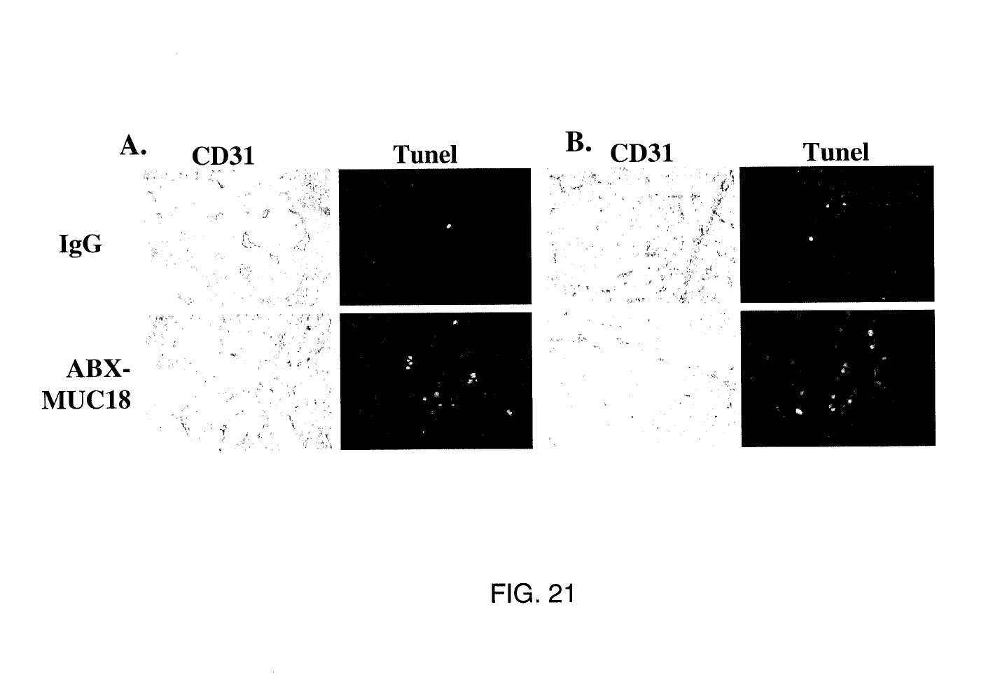

Tumor-associated neovascularization as indicated by microvessel density (MVD) was examined by IHC using anti-CD31 antibody. As shown in FIG. 21, a significant reduction in tumor MVD per field was found after treatment with ABX-MUC18 as compared with control IgG-treated tumors. The mean number of MVD was (8.1±5.1) in ABX-MUC18-treated A375SM tumors; and (201±3.3) in ABX-MUC18-treated WM2664 tumors. In contrast, the mean number of MVD was (22.2±3.1) and (36.1±5.8) for control IgG-treated A375SM and WM2664 tumors, respectively. Moreover, the number of TUNEL-positive tumor cells was inversely correlated with MVD in the studied tumors. The number of tumor cells undergoing apoptosis was higher in the ABX-MUC18-treated animals than in tumors derived from control IgG-treated mice (FIG. 21). These data indicate that ABX-MUC18 treatment significantly decreased tumor-associated neovascularization and subsequently increased apoptosis of tumor cells.

### Example 27

### ABX-MUC18 Interferes with Vessel-Like Tube Formation by HUVEC

HUVEC express the MUC18 cell adhesion molecule, suggesting that MUC18 may play a role in the maturation process of vascular endothelial cells.

Briefly, HUVEC were pretreated with 100 μg/ml ABX-MUC18, 100 μg/ml control IgG, or medium (CMEM) alone for 4 days and then plated on Matrigel to induce vessel-like tube formation. The basement membrane-like substrate (Matrigel) induces HUVEC to rapidly form vessel-like tubes in vitro. Pictures were captured with bright-field microscopy.

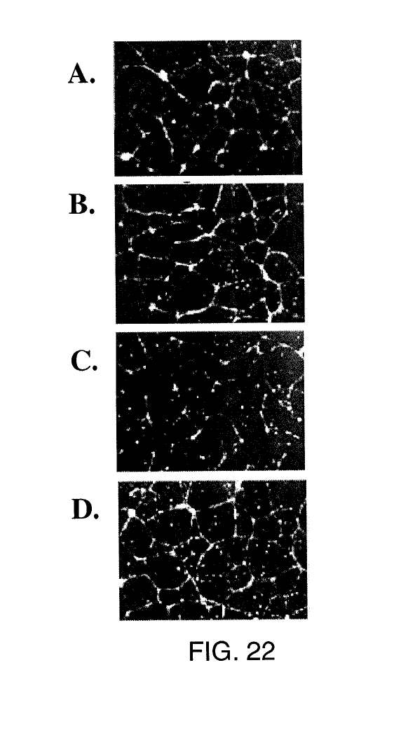

As shown in FIG. 22, CMEM-pretreated HUVEC formed lumen-like structures and anastomosing tubes with multicentric junctions (FIG. 22A). Similar endothelial cell structural morphogenesis occurred in IgG-pretreated HUVEC (FIG. 22B). In contrast, vessel-like tube formation was dramatically disrupted in HUVEC pretreated with ABX-MUC18 (FIG. 22C). When ABX-MUC18 was added to the cultures after tube formation by HUVEC had occurred, the morphogenesis of preexisting vessel-like tubes was not altered (FIG. 22D).

Thus, ABX-MUC18 directly inhibited de novo formation of capillary-like networks but not pre-existing tubule networks.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the composition, methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Reference

The following literature citations as well as those cited above are incorporated in pertinent part by reference herein for the reasons cited in the above text.

U.S. Pat. No. 5,399,363

U.S. Pat. No. 5,466,468

U.S. Pat. No. 5,543,158

U.S. Pat. No. 5,641,515

U.S. Pat. No. 6,150,584

WO 98/24893

Bar-Eli, M. Cancer and Metas. Rev., 18:4358-4369, 1999.

Bar-Eli, M. J. Cell Physiol., 173:275-278, 1997.

Bar-Eli, M. Pathobiology, 67:12-18, 1999.

Bowie et al. Science 253:164, 1991.

Erlandsson, Cancer Genet. Cytogenet, 104:1-18, 1998.

Fidler, I. J. In: Balch, C. M., Houghton, A. N., Milton, G. W., Sober, A. J., Soong, S. J., eds.

Cutaneous melanoma. Philadelphia: J. B. Lippincott Company, Vol, 2, pp 112-129, 1992.

Fidler, I. J. Cancer Res. 50: 6130-6138, 1990.

Fidler and Kripke et al. Science, 197:893-895, 1977.

Gertig and Hunter, Semin. Cancer Biol., 8(4):285-298, 1997.

Goding, 1986, In: Monoclonal Antibodies: Principles and Practice, pp. 60-61 and 71-74.

Griswold, D. E. et al. J. Invest. Dermatol., 97:1019-1023, 1991.

Gutman, M. et al. Anticancer Res., 14:1759-1766, 1994.

Herlyn, M. Cancer Met. Rev., 9:101-112, 1990.

Huang, S. et al. Clin. Cancer Res. 2:1969-1979, 1996.

Jakobovits et al., Proc Natl Acad Sci USA., 90:2551-5, 1993.

Jean, D. et al., Cancer Res., 56:254-258, 1996.

Jones et al., Nature 321:522-5, 1986.

Kerbel, R. S. Am. J. Pathol., 141:519-524, 1992.

Kobayashi, H. S. et al., Proc. Natl. Acad. Sci. USA, 90: 3294-3298, 1993.

Kohler and Milstein, Eur. J. Immunol., 6:511-519, 1976.

Kolmel, J. Neurooncol., 38:121-125, 1998.

Kozbor, J. Immunol. 133:3001-5, 1984.

Lu and Kerbel, et al. Curr. Opin. Oncol., 6:212-220,1994.

Luca, M. et al., Melanoma Res., 3:35-41, 1993.

Luca, M. et al. Int. J. Oncol., 3:19-22, 1993.

Luca, M. et al., Am. J. Pathol., 151:1105-1113, 1997.

Luca, M. et al., Oncogene, 11:1399-1402, 1995.

Magi-Galluzzi et al., Anal. Quant. Cytol. Histol., 20:343-350, 1998.

Mangray and King, Front Biosci., 3:D1148-1160, 1998.

Mastrangelo et al., In: Cancer Principles and Practice of Oncology. Philadelphia: J. B. Lippincott Company, 2:1371-1422, 1985.

Matte, S. et al. Int. J. Cancer, 56:853-857, 1994.

Mayer, Radiat Oncol Investig. 6:281-8, 1998.

McCafferty et al., Nature 348:552-4, 1990.

Mumby and Walter, Cell Regul., 2:589-598, 1991.

Natoli et al., Biochem. Pharmacol., 56(8):915-920, 1998.

Ohara, Acta Oncol. 37:471-4, 1998.

O'Rourke and Altman et al. Ann. Surg. 217:2-5, 1993.

Price, J. E., et al., Cancer Res. 46:5172-5178, 1986.

Radinsky, R. Semin. Cancer Biol., 2:169-177, 1991.

Remington's Pharmaceutical Sciences, 15th Edition, pages 1035-1038 and 1570-1580

Riechman et al., Nature, 332:323-7, 1988.

Rofstad, E. K., et al., Cell Tissue Kinet., 19: 205-216, 1986.

Singh et al., Cancer Res., 55:3669-3674, 1995.

Solyanik et al., Cell Prolif:, 28:263-278, 1995.

Stokke et al., Cell Prolif, 30(5):197-218, 1997.

Verhoeyen et al., Science;239(4847):1534-6, 1988.

Verschragen, C. E. et al., Anticancer Res., 11:529, 1991.

Xie, S. et al., Cancer Res., 57:2295-2303, 1997.

Xie, S. et al., Oncogene, 15:2069-2076, 1997.

What is claimed is:

1. A method of inhibiting hyperproliferative cell growth comprising administering to a patient an effective amount of a humanized anti-interleukin-8 antibody composition, wherein the composition modulates interleukin-8 activity thereby inhibiting hyperproliferative cell growth.

2. The method of claim 1, wherein the humanized antibody is a fully humanized.

3. The method of claim 1, wherein the cell is a tumor cell.

4. The method of claim 2, wherein the tumor cell is a melanoma cell, a bladder cancer cell, a breast cancer cell, a lung cancer cell, a colon cancer cell, a prostate cancer cell, a liver cancer cell, a pancreatic cancer cell, a stomach cancer cell, a testicular cancer cell, a brain cancer cell, an ovarian cancer cell, a lymphatic cancer cell, a skin cancer cell, a brain cancer cell, a bone cancer cell, or a soft tissue cancer cell.

5. The method of claim 4, wherein the cell is a melanoma cell.

6. The method of claim 4, wherein the cell is a bladder cancer cell.

7. The method of claim 1, wherein the antibody is ABX-IL8.

8. The method of claim 1, wherein the patient is a mammal.

9. The method of claim 8, wherein the patient is a human.

10. A method of inhibiting hyperproliferative cell growth comprising administering to a patient an effective amount of a fully humanized anti-interleukin-8 antibody composition in combination with an antitumor agent, wherein the composition modulates interleukin-8 activity thereby inhibiting hyperproliferative cell growth.

11. The method of claim 10, wherein the antitumor agent is a chemotherapeutic drug.

12. The method of claim 10, wherein the antitumor agent is an anti-angiogenic agent.

13. A method of inhibiting hyperproliferative cell growth comprising administering to a patient an effective amount of an anti-melanoma cell adhesion molecule (MCAM/MUC18) antibody composition, wherein the composition modulates MUC18 activity thereby inhibiting hyperproliferative cell growth.

14. The method of claim 13, wherein the antibody is a monoclonal antibody.

15. The method of claim 14, wherein the antibody is humanized.

16. The method of claim 14, wherein the antibody is fully humanized.

17. The method of claim 16, wherein the antibody is ABX-MUC18.

18. The method of claim 17, wherein the ABX-MUC18 antibody is MUC18:A15 6.9, MUC18:A15 6.11 or MUC18:A153.19.

19. The method of claim 13 further comprising administering to the patient a chemotherapeutic drug.

20. The method of claim 13 further comprising administering to the patient a anti-angiogenic agent.

21. The method of claim 17 further comprising administering to a patient a second antibody preparation.

22. The method of claim 21, wherein the second antibody composition is a fully humanized anti-interleukin-8 antibody composition.

23. The method of claim 22, wherein the second antibody is ABX-IL8.

24. A method of inhibiting angiogenesis in a tumor comprising administering to a patient an effective amount of a humanized anti-interleukin-8 antibody composition, wherein the composition modulates interleukin-8 activity thereby inhibiting angiogenesis.

25. The method of claim 24, wherein the humanized anti-interleukin-8 antibody is fully humanized.

26. The method of claim 24 further comprising administering to the patient a humanized anti-MUC18 antibody composition.

27. The method of claim 26, wherein the humanized anti-MUC18 antibody is fully humanized.

28. The method of claim 24, wherein the tumor is further defined as cancer.

29. The method of claim 28, wherein the cancer is melanoma, bladder, non-small cell lung, small cell lung, lung, hepatocarcinoma, retinoblastoma, astrocytoma, glioblastoma, neuroblastoma, head, neck, breast, pancreatic, gum, tongue, prostate, renal, bone, testicular, ovarian, mesothelioma, cervical, gastrointestinal lymphoma, brain, or colon cancer.

30. The method of claim 29, wherein the cancer is melanoma.

31. The method of claim 29, wherein the cancer is bladder cancer.

32. A method of inhibiting metastasis of a tumor comprising administering to a patient an effective amount of a humanized anti-interleukin-8 antibody composition, wherein the composition modulates interleukin-8 activity thereby inhibiting metastasis.

33. The method of claim 32, wherein the humanized anti-interleukin-8 antibody is fully humanized.

34. The method of claim 32, wherein the tumor is further defined as cancer.

35. The method of claim 34, wherein the cancer is melanoma, bladder, non-small cell lung, small cell lung, lung, hepatocarcinoma, retinoblastoma, astrocytoma, glioblastoma, neuroblastoma, head, neck, breast, pancreatic, gum, tongue, prostate, renal, bone, testicular, ovarian, mesothelioma, cervical, gastrointestinal lymphoma, brain, or colon cancer.

36. The method of claim 35, wherein the cancer is a melanoma.

37. The method of claim 35, wherein the cancer is a bladder cancer.

38. The method of claim 32 further comprising administering to the patient a humanized anti-MUC18 antibody composition.

39. The method of claim 38, wherein the humanized anti-MUC18 antibody is fully humanized.

40. A method of inhibiting metastasis of a tumor comprising administering to a patient an effective amount of a humanized anti-MUC18 antibody composition, wherein the composition modulates MUC18 activity thereby inhibiting metastasis.

41. The method of claim 40, wherein the humanized anti-MUC18 is fully humanized.

42. The method of claim 40, wherein the tumor is further defined as cancer.

43. The method of claim 42, wherein the cancer is melanoma, bladder, non-small cell lung, small cell lung, lung, hepatocarcinoma, retinoblastoma, astrocytoma, glioblastoma, neuroblastoma, head, neck, breast, pancreatic, gum, tongue, prostate, renal, bone, testicular, ovarian, mesothelioma, cervical, gastrointestinal lymphoma, brain, or colon cancer.

44. The method of claim 43, wherein the cancer is a melanoma.

45. The method of claim 43, wherein the cancer is a bladder cancer.

46. The method of claim 40 further comprising administering to the patient a humanized anti-interleukin-8 antibody composition.

47. The method of claim 46, wherein the humanized anti-interleukin-8 antibody is fully humanized.

48. A method of inhibiting angiogenesis in a tumor comprising administering to a patient an effective amount of a humanized anti-MUC18 antibody composition, wherein the composition modulates MUC18 activity thereby inhibiting angiogenesis.

49. The method of claim 48, wherein the humanized anti-MUC18 is fully humanized.

50. The method of claim 48 further comprising administering to the patient a humanized anti-interleukin-8 antibody composition.

51. The method of claim 50, wherein the humanized anti-interleukin-8 antibody is fully humanized.

52. A method of inhibiting hyperproliferative cell growth comprising administering to a patient a combination of an effective amount of a fully humanized anti-MUC18 antibody composition and a fully humanized anti-interleukin-8 antibody composition, wherein the combination modulates MUC18 and interleukin-8 activity thereby inhibiting hyperproliferative cell growth.

53. The method of claim 52, wherein the cell is a tumor cell.

54. The method of claim 53, wherein the tumor cell is a melanoma cell, a bladder cancer cell, a breast cancer cell, a lung cancer cell, a colon cancer cell, a prostate cancer cell, a liver cancer cell, a pancreatic cancer cell, a stomach cancer cell, a testicular cancer cell, a brain cancer cell, an ovarian cancer cell, a lymphatic cancer cell, a skin cancer cell, a brain cancer cell, a bone cancer cell, or a soft tissue cancer cell.

55. The method of claim 54, wherein the tumor cell is a melanoma cell.

56. The method of claim 54, wherein the tumor cell is a bladder cancer cell.

57. A method of treating a hyperproliferative disease comprising administering to a patient an effective amount of a fully humanized anti-interleukin-8 antibody composition in combination with chemotherapy, immunotherapy, surgery, or radiotherapy.

58. The method of claim 57, wherein the chemotherapy comprises a DNA damaging agent.

59. The method of claim 58, wherein the DNA damaging agent is gamma-irradiation, X-rays, UV-irradiation, microwaves, electronic emissions, adriamycin, 5-fluorouracil (5FU), etoposide (VP-16), campotothecin, actinomycin-D, mitomycin C, cisplatin (CDDP), or hydrogen peroxide.

60. The method of claim 57, wherein the immunotherapy comprises an anti-MUC18 antibody.

61. The method of claim 60, wherein the antibody is fully humanized.

62. The method of claim 61, wherein antibody is ABX-MUC18.

63. The method of claim 57, wherein the hyperproliferative disease is cancer.

64. The method of claim 63, wherein the cancer is melanoma, bladder, non-small cell lung, small cell lung, lung, hepatocarcinoma, retinoblastoma, astrocytoma, glioblastoma, neuroblastoma, head, neck, breast, pancreatic, gum, tongue, prostate, renal, bone, testicular, ovarian, mesothelioma, cervical, gastrointestinal lymphoma, brain, or colon cancer.

65. The method of claim 64, wherein the cancer is melanoma.

66. The method of claim 64, wherein the cancer is bladder cancer.

67. A method of treating a hyperproliferative disease comprising administering to a patient an effective amount of a fully humanized anti-MUC18 antibody composition in combination with chemotherapy, immunotherapy, surgery, or radiotherapy.

68. The method of claim 67, wherein the chemotherapy comprises a DNA damaging agent.

69. The method of claim 68, wherein the DNA damaging agent is gamma-irradiation, X-rays, UV-irradiation, microwaves, electronic emissions, adriamycin, 5-fluorouracil (5FU), etoposide (VP-16), campotothecin, actinomycin-D, mitomycin C, cisplatin (CDDP), or hydrogen peroxide.

70. The method of claim 67, wherein the immunotherapy comprises an anti-interleukin-8 antibody.

71. The method of claim 70, wherein the antibody is fully humanized.

72. The method of claim 71, wherein antibody is ABX-IL8.

73. The method of claim 67, wherein the hyperproliferative disease is cancer.
